(19) United States
(12) Patent Application Publication
(10) Pub. No.: US 2013/0253213 A1
(43) Pub. Date: Sep. 26, 2013

(54) METHOD FOR PRODUCING CYANOACETIC ACID, METHOD FOR PRODUCING CYANOACETIC ACID DERIVATIVE AND METHOD FOR PRODUCING METAL CONTAINING COMPOUND

(71) Applicants: Dai IKEMIZU, Tokyo (JP); Ryohei IWAMOTO, Tokyo (JP); Keiko ISHIDAI, Tokyo (JP); Kimihiko OOKUBO, Tokyo (JP)

(72) Inventors: Dai IKEMIZU, Tokyo (JP); Ryohei IWAMOTO, Tokyo (JP); Keiko ISHIDAI, Tokyo (JP); Kimihiko OOKUBO, Tokyo (JP)

(73) Assignee: Konica Minolta Business Technologies, Inc., Tokyo (JP)

(21) Appl. No.: 13/841,940

(22) Filed: Mar. 15, 2013

(30) Foreign Application Priority Data
Mar. 26, 2012 (JP) 2012-070371

(51) Int. Cl. C07C 253/30; C07F 1/08

(52) U.S. Cl. CPC C07C 253/30; C07F 1/08
USPC 556/117; 558/443; 558/398

(57) ABSTRACT

Provided is a method for producing cyanoacetic acid in a hydrolysis reaction of a predetermined cyanoacetate in the presence of an acid catalyst. Further, are provided methods for producing a cyanoacetic acid derivative and a metal containing compound by using the produced cyanoacetic acid as a staring material. Herein, the method for producing cyanoacetic acid enables the content of a malonic acid byproduct generated in the hydrolysis reaction to be greatly lowered, allowing the produced cyanoacetic acid to be used as a starting material without any purification treatments. Those advantageous effects result in the great improvement in the purity and yields of the cyanoacetic acid derivative and the metal containing compound produced by said cyanoacetic acid. Accordingly, the above mentioned methods make it possible to produce cyanoacetic acid, the cyanoacetic acid derivative and the metal containing compound, as excellent in the productivity and economical efficiency.

### CROSS REFERENCE TO RELATED APPLICATIONS

This application claims the foreign priority benefit under Title 35, United States Code, 119 (a)-(d) of Japanese Patent Application No. 2012-070371, filed on Mar. 26, 2012 in the Japan Patent Office, the disclosure of which is herein incorporated by reference in its entirety.

### BACKGROUND OF THE INVENTION

1. Technical Field

The present invention relates to a method for producing cyanoacetic acid via hydrolysis of a cyanoacetate with a predetermined chemical formula in the presence of an acid catalyst. Further, the present invention relates to a method for producing a cyanoacetic acid derivative from the produced cyanoacetic acid, and a metal containing compound from the produced cyanoacetic acid derivative.

2. Description of the Prior Art

Cyanoacetic acid and a derivative thereof have been known as a synthetic intermediate to produce an organic reagent such as an adhesive, a medicine and an agricultural chemical. The synthetic methods using cyanoacetic acid and a derivative thereof are known, including a method for undergoing a reaction of chloroacetic acid, bromoacetic acid or a corresponding sodium salt thereof with sodium cyanide (for example, see JPS57-35539), a method for oxidizing cyanoacetaldehyde acetal, cyanoacetaldehyde or cyanoacetaldehyde hydrate under pressurized oxygen (for example, see JPH07-233135), a method for producing cyanoacetic acid by treating ethylene cyanohydrin with microorganisms (for example, see JPS62-32891), and a method for hydrolyzing ethyl cyanoacetate in the presence of nitric acid (for example, see WO2010/46780).

Further, a method for hydrolyzing ethyl cyanoacetate in the presence of hydrochloric acid is described in the J. Org. Chem., 1994, vol. 59, pp. 291-296. Also, a method for hydrolyzing methyl cyanoacetate using sodium oxide is described in the Can. Journal of Chemistry, 1980, vol. 58, pp. 1281-1294.

Moreover, examples of adding a metal containing compound to a dye are reported in order to improve the stability of the dye used in an electronic photography toner (for example, see JP2009-221125, JP2009-222B47, JP2010-072286, and WO20H/010509).

However, the above mentioned conventional techniques have the following drawbacks.

That is, the method described in JPS57-355539 has a major operational drawback that soda cyanide dangerous in handling thereof has to be used in the reaction. Also, the method described in JPH07-233135 has a drawback that it is needed to install special equipment to prepare a pressurized condition. Further, the method described in JPS62-32891 has a drawback that it is needed to prepare a special condition of treating microorganisms as well as a productivity drawback. Moreover, the method described in WO2010/46780 has a drawback that the synthetic yield is low around in 70%, which has to be more improved.

According to the J. Org. Chem., 1994, vol. 59, pp. 291-296, a hydrolysis reaction is described therein, while any yields and purity of the product are not described therein. Further, according to the Can. Journal of Chemistry, 1980, vol. 58, pp. 1281-1294, there are still drawbacks remaining on removal of the produced salts and suppression of the byproduct yield, the drawbacks having to be improved.

Recently, the amounts of the cyanoacetic acid supplied in Japan have been decreased, resulting in the difficulty to obtain cyanoacetic acid at low cost and high quality.

According to the patent documents of JP2009-221125, JP2009-222847, JP2010-072286 and WO2011/010509, most of the metal containing compounds described therein are produced from cyanoacetic acid as a starting material. This results in the necessity for stably supplying cyanoacetic acid with high quality. However, due to the recent circumstance as mentioned above, the stable supply of cyanoacetic acid with high quality and at low cost comes to be more difficult. Eventually, this causes a strong demand to immediately address the above mentioned issues.

Herein, the present inventors have made significant efforts to solve those drawbacks. Accordingly, it was found that cyanoacetic acid was able to be easily produced by hydrolyzing a cyanoacetate in the presence of a general and cheap acid catalyst. On the other hand, it also turned out that the quality and yield of the product obtained after the hydrolysis step were markedly influenced by malonic acid generated as a byproduct during the production of cyanoacetic acid.

### SUMMARY OF THE INVENTION

From the viewpoint of solving the drawbacks as described above, the present invention has been developed. Therefore, the present invention is directed to a method for producing cyanoacetic acid via a hydrolysis reaction of a predetermined cyanoacetate in the presence of an acid catalyst, the method enabling the content of a malonic acid byproduct generated in a hydrolysis reaction of the cyanoacetate to be greatly suppressed.

Further, the present invention is directed to a method for producing a cyanoacetic acid derivative from the produced cyanoacetic acid as a starting material, and a method for producing a metal containing compound from the produced cyanoacetic acid derivative, both in an economical manner.

To achieve at least one of the above mentioned objects, a method for producing cyanoacetic acid, comprising the step of hydrolyzing a cyanoacetate of a formula (1) in the presence of an acid catalyst in a reaction mixture thereby to obtain cyanoacetic acid together with an alcohol and a malonic acid byproduct. Herein, the cyanoacetate of the formula (1) is included in 0.5 to 5 mol %, preferably in 0.5 to 2.0 mol % with respect to the produced cyanoacetic acid in the reaction mixture, when the hydrolysis reaction is completed. The alcohol is included in 0.5 to 60 mol %, preferably in 0.5 to 20 mol % with respect to the produced cyanoacetic acid in the reaction mixture, when the hydrolysis reaction is completed. Further, the malonic acid generated as a byproduct is included in 1.0 mol % or less with respect to the produced cyanoacetic acid in the reaction mixture, when the hydrolysis reaction is completed,

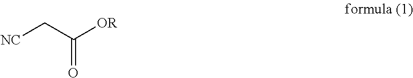

[where R represents an ethyl group or a methyl group].

Further, it is preferable that the above mentioned acid catalyst is selected from a group of sulfuric acid, hydrochloric acid, acetic acid, cyanoacetic acid, phosphoric acid, and p-toluenesulfonic acid.

Further, it is also preferable that the acid catalyst is used in 0.2 to 10 mol %, more preferably in 0.2 to 2.0 mol % with respect to the cyanoacetate of the formula (1) in the hydrolysis reaction.

Furthermore, it is more preferable that the method for producing cyanoacetic acid further includes the step of removing the alcohol produced in the hydrolysis reaction via distillation.

### BRIEF DESCRIPTION OF THE DRAWINGS

FIG. 1 is a schematic diagram showing a cross-section of a toner particle which is produced by dispersing coloring micro particles in a thermoplastic resin according to an embodiment of the present invention.

FIG. 2 is a schematic diagram showing a cross-section of a coloring micro particle having a core shell structure which is formed by covering a core with a shell resin according to an embodiment of the present invention.

### MODES FOR CARRYING OUT THE INVENTION

Hereinafter, embodiments of the present invention will be described in detail.

<<Method for Producing Cyanoacetic Acid>>

A method for producing cyanoacetic acid in an embodiment of the present invention comprises the step of hydrolyzing a cyanoacetate of the following formula (1) in the presence of an acid catalyst, thereby to produce target cyanoacetic acid,

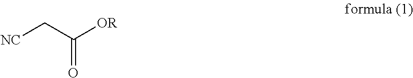

[where R represents an ethyl group or a methyl group].

Herein, at the end point of the reaction, the reaction mixture includes the cyanoacetate of the formula (1) in 0.5 to 5 mol % with respect to the produced cyanoacetic acid, the alcohol produced in the hydrolysis reaction in 0.5 to 60 mol % with respect to the produced cyanoacetic acid, and malonic acid in the content of 1.0 mass % or less with respect to the produced cyanoacetic acid.

More specifically, the method for producing cyanoacetic acid in the embodiment of the present invention is conducted by mixing the cyanoacetate of the formula (1), a solvent, and an acid catalyst, thereby to hydrolyze the cyanoacetate in the presence of the acid catalyst. The hydrolysis step further includes the step of removing the produced alcohol by distillation.

[Solvent]

Water is used as a solvent in the hydrolysis step of the cyanoacetate of the formula (1). Herein, it should be noted that if solubility of the cyanoacetate in water is relatively low, a water-soluble solvent may be used as the reaction solvent.

Such a solvent includes, for example, alcohols such as methanol, ethanol, and propanol or the like; glycols such as ethylene glycol, propylene glycol, glycerin, methyl cellosolve, and ethyl cellosolve or the like; ethers such as ethylene glycol dimethyl ether, tetrahydrofuran, and dioxane or the like; other solvents such as N,N-dimethylformamide, N,N-dimethylacetamide, acetonitrile, propionitrile, dimethyl sulfoxide, 1,3-dimethyl-2-imidazolidinone, and sulfolane or the like.

When a water-soluble solvent is used as mixed with water, the rate of the solvent to water is in the range from 1 to 100 wt %, preferably 1 to 50 wt %, more preferably 1 to 10 wt %. Further, the volume of the solvent used in the hydrolysis of the cyanoacetate of the formula (1) to the volume of the cyanoacetate used in the hydrolysis is preferably 3-fold to 10-fold, more preferably 4-fold to 8-fold, and more preferably 4-fold to e-fold.

[Acid Catalyst]

An acid catalyst used in the hydrolysis of the cyanoacetate of the formula (1) is selected from a group of inorganic acids such as sulfuric acid, nitric acid, hydrochloric acid, hydrobromic acid, hydrofluoric acid, phosphoric acid, and boric acid; and organic acids such as acetic acid, trifluoro acid, p-toluenesulfonic acid, methansulfonic acid, and cyanoacetic acid or the like. Herein, as mentioned hereinafter, in order to avoid contamination in the next step, that is, in the dehydration condensation, and further in order to suppress production of a byproduct, preferable acid catalysts include sulfuric acid, hydrochloric acid, acetic acid, phosphoric acid, p-toluenesulfonic acid, and cyanoacetic acid, more preferable acid catalysts include phosphoric acid, p-toluenesulfonic acid, and cyanoacetic acid, the most preferable acid catalyst includes p-toluenesulfonic acid.

The preferable rate of the acid catalyst used in the hydrolysis of the cyanoacetate to the cyanoacetate of the formula (1) is preferably set in the range from 0.2 to 10 mol %, more preferably 0.2 to 5 mol %, and most preferably 0.2 to 1 mol %

[Remaining Rate of Cyanoacetate and Production Rate of Alcohol]

In an embodiment of the present invention, the remaining rate of the cyanoacetate of the formula (1) to the produced cyanoacetic acid is in the range from 0.5 to 5 mol %. Further, the production rate of the alcohol to the produced cyanoacetic acid yielded in the hydrolysis reaction is in the range from 0.5 to 60 mol %.

Here, examinations of the inventors demonstrated that in the hydrolysis step of the cyanoacetate in the embodiment of the present invention, the acid catalyst facilitated the hydrolysis reaction, and the produced cyanoacetic acid in the hydrolysis was further hydrolyzed thereby to produce malonic acid. Hereby, in order to suppress the further hydrolysis of the produced cyanoacetic acid, it was essential to take advantage of the different hydrolysis rates between the 2 substrates (that is, the cyanoacetate and the produced cyanoacetic acid), together with the equilibrium of the hydrolysis. In short, the hydrolysis of the cyanoacetate proceeds in a preferred manner at the earlier stage of the hydrolysis step, while the hydrolysis of the produced cyanoacetic acid proceeds in a preferred manner at the latter stage of the hydrolysis step. Note the hydrolysis reaction of the cyanoacetate is an equilibrium, reaction. Accordingly, it comes to be possible to greatly suppress the production of malonic acid by balancing the control in a removing volume of the produced alcohol via distillation and the consumption rate of the cyanoacetate so as not to completely hydrolyze the remaining cyanoacetate.

Therefore, at the end point of the hydrolysis reaction, the cyanoacetate represented by the formula (1) per the produced cyanoacetic acid is set to be included in the range from 0.5 to 5 mol %. Further, the alcohol produced in the hydrolysis reaction per the produced cyanoacetic acid is set to be included in the range from 0.5 to 60 mol %. If the contents of cyanoacetic acid and the alcohol are set in the above mentioned range, this allows the production of malonic acid to be markedly suppressed.

Then, in order to control the content of the alcohol produced in the hydrolysis reaction in the range from 0.5 to 60 mol %, a step of removing the alcohol by distillation (or a step of distilling the alcohol) is conducted in the hydrolysis step.

### <Alcohol Removing Step by Distillation>

It is preferable to use a Claisen flask or a Dean-Stark apparatus for the distillation process in the method for removing the alcohol produced in the hydrolysis of the cyanoacetate of the formula (1).

The method for removing the alcohol via distillation is not limited to a specific procedure as long as the hydrolysis in the reaction mixture proceeds with the removal of the produced alcohol via distillation. However, as mentioned above, it should be noted that at the end of the hydrolysis reaction the alcohol produced in the reaction to the yielded cyanoacetic acid in the reaction mixture is set to be included in the range from 0.5 to 60 mol %, preferably from 0.5 to 40 mol %, more preferably from 0.5 to 20 mol %.

Herein, the removal of the alcohol via distillation may be conducted in a continuous manner. Alternatively, the removal operation may be conducted in an intermittent manner. That is, the hydrolysis reaction is conducted under a reflux condition without removing alcohol via distillation until a certain degree of the reaction progress is checked. Based on the reaction check, a predetermined amount of the alcohol comes to be removed via distillation. Then, the refluxing operation is performed again. Otherwise, the removal of the alcohol via distillation may be conducted by repeating the above mentioned operations. In such a case, preferably a volume of the removed alcohol in one operational cycle is set in the range from 5 to 20% with respect to the total volume of the solvent. In such a case, a desirable volume of water may be added into the reaction mixture in proportion to the removed volume of the alcohol via distillation.

Herein, the certain degree of the reaction progress as mentioned above means that the content of the produced cyanoacetic acid in the reaction mixture comes to be higher than 30 mol %, preferably higher than 40 mol %, and more preferably higher than 50 mol %. Note the content of the produced cyanoacetic acid in the reaction mixture can be calculated by referring to the simple area ratio obtained in the gas chromatography analysis together with the calibration curve data in the gas chromatography analysis created by using the commercially available cyanoacetate and cyano acetic acid.

A reaction time needed for the hydrolysis step of the cyanoacetate of the formula (1) is not specifically limited as long as the hydrolysis reaction smoothly proceeds. However, a shorter reaction time is preferable so as to suppress the further hydrolysis of the produced cyanoacetic acid. More specifically, preferably the reaction time is set in the range from 5 to 20 hrs, more preferably from 5 to 10 hrs. and most preferably from 5 to 7 hrs.

In an embodiment of the present invention, it is possible to variously treat an aqueous solution of cyanoacetic acid obtained after the hydrolysis reaction. For example, as described in JPS57-35539, the resultant aqueous solution may be condensed under a reduced pressure and used in the next step. In such a case, it is preferable to set the condensed temperature at 80° C. or less, more preferably in the range form 40 to 80° C., and most preferably in the range from 40 to 60° C. When the aqueous solution is condensed under a reduced pressure, preferably a condensed degree of cyanoacetic acid is set in the range from 50 to 95%, and more preferably from 55 to 90%.

Herein, when an aqueous solution of cyanoacetic acid in a highly condensed state is cooled, crystals thereof come to be precipitated. Therefore, after removal of an unnecessary volume of water via decantation, centrifugal separation, or filtration separation from the precipitated crystals, the resultant crystals can be used as they are in the next step.

A method for detecting the remaining cyanoacetate in the reaction mixture includes ion chromatography, NMR, gas chromatography, HS-GC, mass spectrometry, HPLC, GC-MS, capillary electrophoresis, preparative GPC or the like. In a preferable method, a rate of the cyanoacetate to cyanoacetic acid has been calculated beforehand by using a calibration curve obtained in gas chromatography. Then, the reaction is to be terminated when a remaining content of the cyanoacetate reaches a desired content in the analysis of the reaction solution. For example, the end point of the hydrolysis reaction may be determined at the time when the content of the cyanoacetate is substantially to be 2 mol %, by comparing the remaining contents with the calibration curve data on the cyanoacetate and cyanoacetic acid.

[Content of Malonic Acid: 1.0 Mass % or Less]

The content of malonic acid included in cyanoacetic acid obtained in an embodiment of the present invention is 1.0 mass % or less, preferably 0.8 mass % or less, and more preferably 0.6 mass %, or less.

A method for analyzing malonic acid includes HPLC, capillary electrophoresis, mass spectrometry, preparative GPC, and ion chromatography or the like. A preferable method is a quantitative analysis using ion chromatography. That is, an absolute content of malonic acid in an aqueous solution sample is calculated by using a calibration curve of malonic acid obtained using the analytical standard. Then, the comparison examinations among the respective samples allow the effects in the embodiment of the present invention to be determined.

<<Method for Producing Cyanoacetic Acid Derivative>>

Hereinafter, a method for producing a cyanoacetic acid derivative of the following formula (3) will be explained in detail, which is to be conducted in the next step after the cyanoacetate of the formula (1) has been hydrolyzed.

A method for producing a cyanoacetic acid derivative in an embodiment of the present invention comprises the step of undergoing a reaction of a compound defined in the following formula (2) with cyanoacetic acid produced in the method described hereinbefore, thereby to produce the cyanoacetic acid derivative of the formula (3).

[Cyanoacetic Acid Derivative-1 (Cyanoacetic Acid Derivative of Formula (3))]

Cyanoacetic acid obtained in an embodiment of the present invention is converted to the cyanoacetic acid derivative of the formula (3) by the reaction with the alcohol derivative of the formula (2).

HO—R₁ formula (2)

[where R₁ represents a group having an aromatic hydrocarbon structure including at least 9 carbon atoms].

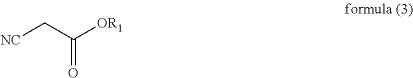

[where R₁ represents the group having the aromatic hydrocarbon structure including at least 9 carbon atoms].

Here, R₁ represents a group having an aromatic hydrocarbon structure including at least 9 carbon atoms.

The group having the aromatic hydrocarbon structure including at least 9 carbon atoms may be defined so that the total number of the carbon atoms in R₁ is 9 or more, and the aromatic hydrocarbon structure is included at an optional position in R₁. Such examples of the aromatic hydrocarbon structure include an aryl group such as a phenyl group and a naphthyl group or the like.

For example, when the aromatic hydrocarbon structure is a phenyl group, R₁ is formed together with an optional group having at least 3 carbon atoms. In such a case, 3 or more substituents each of which has one carbon atom may be combined to form the optional group.

Alternatively, at least one substituent having one carbon atom and at least one substituent having 2 carbon atoms may be combined each other. Preferably the total number of the carbon atoms in R₁ is set in the range from 9 or more to 40 or less, more preferably from 12 or more to 40 or less, and most preferably 14 or more to 30 or less.

The preferable R₁ is defined by the following formula (2′).

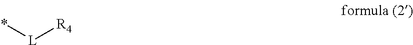

In the formula (2′), “L” represents a group formed by combining a bivalent linking group selected from an alkylene group having 1 to 15 carbon atoms, —SO₂O—, —OSO₂—, —SO₂—, —CO—, —O—, —S—, —SO₂NH—, —NHSO₂—, —CONH—, —NHCO—, —COO—, —OOC—, composed of a single group or a plurality of groups. Herein, “L” is connected to the oxygen atom adjacent to R₁ of the formula (3) at the position represented by the mark of “*”.

Here, “L” may have a substituent including, for example, an alkyl group such as a methyl group, an ethyl group, a propyl group, an isopropyl group, a tert-butyl group, a pentyl group, a hexyl group, an octyl group, a dodecyl group, a tridecyl group, a tetradecyl group, pentadecyl group, cyclopentyl group, and a cyclohexyl group or the like; an alkenyl group such as a vinyl group and an allyl group or the like; an alkynyl group such as an ethynyl group and a propargyl group; an aryl group such as a phenyl group and a naphthyl group; a heteroaryl group such as a furyl group, a thienyl group, a pyridyl group, pyridazyl group, a pyrimidyl group, a pyrazyl group, a triazyl group, an imidazolyl group, a pyrazolyl group, a thiazolyl group, a benzimidazolyl group, a benzoxazolyl group, a quinazolyl group, and a phtharazyl group; a heterocyclic group such as a pyrrolidyl group, an imidazolidinyl group, a morpholyl group, and an oxazolidyl group or the like; an alkoxy group such as a methoxy group, an ethoxy group, a propyloxy group, a pentyloxy group, a hexyloxy group, an octyloxy group, a dodecyloxy group, a cyclopentyloxy group, and a cyclohexyloxy group or the like; an aryloxy group such as a phenoxy group and a naphthyloxy group; an alkylthio group such as a methylthio group, an ethylthio group, a propylthio group, a pentylthio group, a hexylthio group, an octylthio group, a dodecylthio group, a cyclopentylthio group, and a cyclohexylthio group or the like; an arylthio group such as a phenylthio group and a naphthylthio group; an alkoxycarbonyl group such as a methyloxycarbonyl group, an ethyloxycarbonyl group, a butyloxycarbonyl group, an octyloxycarbonyl group, a dodecyloxycarbonyl group or the like; an aryloxycarbonyl group such as a phenyloxycarbonyl group and a naphthyloxycarbonyl group; a sulfamoyl group such as an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphtylaminosulfonyl group, and a 2-pyridylaminosulfonyl group; an acyl group such as an acetyl group, an ethylcarbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphtylcarbonyl group, a pyridylcarbonyl group; an acyloxy group such as an acetyloxy group, an ethylcarbonyloxy group, a butylcarbonyloxy group, an octylcarbonyloxy group, a dodecylcarbonyloxy group, a phenylcarbonyloxy group; an amide group such as methylcarbonylamino group, an ethylcarbonylamino group, a dimethylcarbonylamino group, a propylcarbonylamino group, a pentylcarbonylamino group, g cyclohexylcarbonylamino group, a 2-ethylhexylcarbonylamino group, an octylcarbonylamino group, a dodecylcarbonylamino group, a phenylcarbonylamino group, and a naphthylcarbonylamino group; a carbamoyl group such as an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecylaminocarbonyl group, a phenylaminocarbonyl group, a naphtylaminocarbonyl group, 2-pyridylaminocarbonyl group or the like; a ureido group such as a methyl ureido group, an ethylureido group, a pentylureido group, a cyclohexylureido group, an octylureido group, a dodecylureido group, a phenylureido group, a naphtylureido group, and a 2-pyridylaminoureido group or the like; a sulfinyl group such as a methylsulfinyl group, an ethylsulfonyl group, a butylsulfonyl group, a cylohexylsulfonyl group, a 2-ethylhexylsulfinyl group, a dodecylsulfinyl group, a phenylsulfinyl group, a naphthylsulfonyl group, and a 2-pyridylsulfonyl group or the like; an alkylsulfonyl group such as a methylsulfonyl group, an ethylsulfonyl group, a butylsulfonyl group, a cylohexylsulfonyl group, a 2-ethylhexylsulfonyl group, and a dodecylsulfonyl group or the like; an arylsulfonyl group such as a phenylsulfonyl group, a naphthylsulfonyl group, and a 2-pyridylsulfonyl group or the like; an amino group such as an amino group, an ethylamino group, a dimethylamino group, a butylamino group, a cyclopentylamino group, a 2-ethylhexylamino group, a dodecylamino group, an anilino group, a naphthylamino group, and a 2-pyridylamino group; a cyano group, a nitro group, and a halogen atom such as a chlorine atom, a bromine atom, a fluorine atom, and an iodine atom or the like. Herein, the above mentioned group may be further substituted by such a group.

A bivalent linking group represented by “L” preferably includes an alkylene group or a group containing an alkylene group. The group containing an alkylene group may include a alkylene group at an optional position within the bivalent linking group represented by “L”. More specifically, the group containing an alkylene group is a group including an alkylene group in the structure of the group formed by one bivalent linking group selected from an alkylene group, —SO₂O—, —OSO₂—, —SO₂—, —CO—, —O—, —S—, —SO₂NH—, —NHSO₂—, —CONH—, —NHCO—, COO— and —OOC—, ox formed by combining a plurality of the above mentioned bivalent linking groups.

R₄ represents an aryl group such as a phenyl group and a naphthyl group.

Hereinafter, specific examples of the bivalent linking group represented by “L” will be shown. However, the embodiment of the present invention is not limited to such specific examples, and various modifications thereof may be performed.

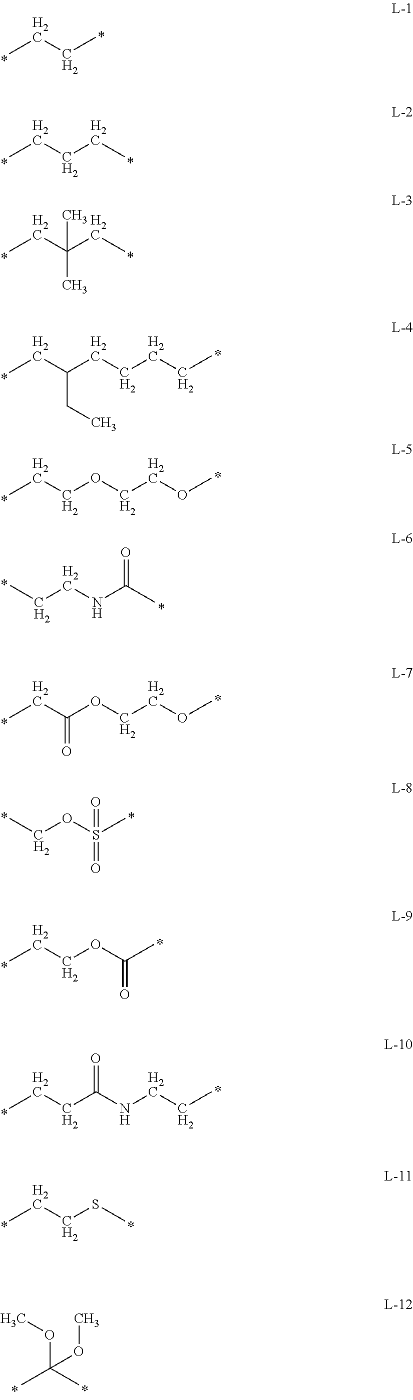

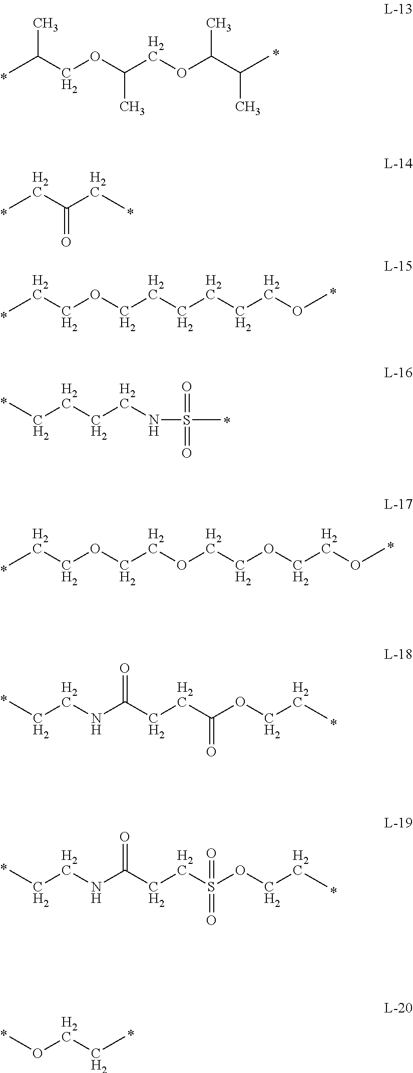

Herein, “L” is bound to the oxygen atom adjacent to R₁ of the formula (1) or R₄ at the position marked as “*”, R₄ represents an aryl group such as a phenyl group and a naphthyl group.

R₁ and R₄ may have a substituent, being the same group as a substituent which is capable of positioned on the above defined “L”.

A preferable substituent on R₁ and R₄ includes an alkyl group, an alkoxy group, an aryloxy group, an alkylthio group, an arylthio group, an alkoxycarbonyl group, an aryloxycarbonyl group, a sulfamoyl group, an acyl group, an acyloxy group, an amide group, a carbamoyl group, an alkylsulfonyl group, an arylsulfonyl group, an amino group, a cyano group, a nitro group and a halogen atom. A more preferable substituent includes an alkyl group, an alkoxy group, an aryloxy group, an alkoxycarbonyl group, an aryloxycarbonyl group, a sulfamoyl group, an acyl group, an acyloxy group, an amide group and a carbamoyl group. The most preferable substituent includes an alkyl group, an alkoxy group, an aryloxy group, an aryloxycarbonyl group, an acyloxy group and an amide group.

Preferably R₄ is a phenyl group, preferably having a substituent thereon. A preferable substituent includes an alkyl group, an alkoxy group, an aryloxy group, an aryloxycarbonyl group, an acyloxy group and an amide group. A more preferable substituent includes an alkyl group and an alkoxy group.

Preferably R₁ or the formula (2′) is a group represented by the following formula (2′-2).

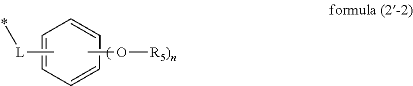

In the formula (2′-2), “L” and represent the same group defined as in the formula (2′). R₅ represents an alkyl group having 8 to 30 carbon atoms and “n” represents an integer in the range from 1 to 3.

Preferably R₅ is an alky group having 12 to 24 carbon atoms, more preferable an alkyl group having 16 to 24 carbon atoms. R₅ may have a substituent, being the same group as a substituent which is capable of being positioned on “L” of the formula (2′). Preferably is a linear alkyl group, and more preferably the group is composed of only a carbon atom and a hydrogen atom. Preferably “n” is equal to 1 or 2, more preferably equal to 1.

The method for producing the compound of the formula (3) is a so-called “esterification”, that is, a condensation reaction between a carboxylic acid and an alcohol. The usage rate of the alcohol derivative of the formula (2) with respect to cyanoacetic acid in preparation of the compound represented by the formula (3) is preferably in the range from 0.7 eq. to 1.0 eq., more preferably form 0.8 eq. to 1.0 eq., most preferably from 0.9 eq. to 1.0 eq.

A solvent used in the condensation reaction between the produced cyanoacetic acid obtained in an embodiment using the method of the present invention and the alcohol derivative of the formula (2) is not specifically limited as long as the condensation reaction smoothly proceeds in the solvent. For example, it is preferable to use a solvent of which boiling point is higher than that of water. Further, preferably the solvent is a hydrocarbon based solvent. Such a hydrocarbon based solvent includes toluene, xylene, mesitylene, octane and nonane or the like. From the viewpoint of saving the cost and collecting a solvent easily, the more preferable solvent includes toluene and xylene.

A volume of the solvent used in the preparation of the compound represented by the formula (3) is preferably 2-fold to 10-fold larger than that of the alcohol derivative represented by the formula (3), more preferably 2-fold to 5-fold larger, most preferably 2-fold to 3-fold larger.

A catalyst used in the preparation of the compound represented by the formula (3) is selected from an inorganic acid such as sulfuric acid, nitric acid, hydrochloric acid, hydrobromic acid, hydrofluoric acid, phosphoric acid and boric acid; and an organic acid such as acetic acid, trifluoroacetic acid, p-toluenesulfonic acid, methansulfonic acid and cyanoacetic acid or the like. Herein, it should be noted that in order to suppress the production of a byproduct, preferably the catalyst is selected from sulfuric acid, hydrochloric acid, phosphoric acid and p-toluenesulfonic acid or the like, more preferably phosphoric acid and p-toluenesulfonic acid, most preferably p-toluenesulfonic acid.

A rate of the catalyst used in the preparation of the compound represented by the formula (3) to the alcohol derivative of the formula (2) is set in the range from 2 to 10 mol %, more preferably from 2 to 5 mol %, most preferably from 2 to 4 mol %.

(Reaction Process)

In the reaction of the alcohol derivative represented by the formula (2) with cyanoacetic acid, the resultant cyanoacetic acid obtained in the previous step (or the hydrolysis step) is included in an aqueous solution. Accordingly, a large amount of water is to be contained in the reaction mixture corresponding to a degree of the concentrate thereof. Generally, the esterification reaction of a carboxylic acid proceeds by removing water produced in the reaction mixture to the outside of the reaction mixture. Further, in the reaction of the present embodiment, production of malonic acid via the hydrolysis of cyanoacetic acid is not negligible similarly to the state of the previous step.

From the viewpoint as mentioned above, the present inventors have focused on the improvement of the reaction conditions. Eventually, the inventors found out that malonic acid produced in the previous step and the present step (that is, the step of producing the cyanoacetic acid derivative of the formula (3)), caused the condensation reaction with the alcohol derivative of the formula (2) to produce a malonic acid derivative of the following formula (3′), like cyanoacetic acid.

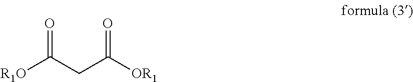

[where R₁ represents the group having the aromatic hydrocarbon structure containing 9 or more carbon atoms].

That condensation reaction was found to decrease the yields and purity of the target products in the present step and the next step (that is, the step of producing a cyanoacetic acid derivative of the formula (4)

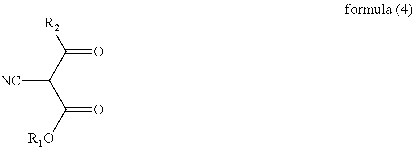

[where R₁ represents the group having the aromatic hydrocarbon structure containing 9 or more carbon atoms, and R₁ represents the alkyl group].

Accordingly, in order to suppress the undesired reaction progress and the byproduct production, it is effective to efficiently remove i) water derived from the previous step and ii) water produced in the present step.

In an embodiment of the present invention, preferably a method for removing water via distillation in the preparation of the compound represented by the formula (3) includes a process of removing water via distillation by using a Claisen flask or a Dean-Stark apparatus (or an esterification tube). Such a method for removing water in the preparation of the compound represented by the formula (3) is not specifically limited as long as the condensation reaction in the reaction mixture proceeds in association with the consumption of the starting material (that is, cyanoacetic acid) and the removal of the produced water.

Herein, it is preferable to shorten the time for removing water contained in the reaction mixture due to the above mentioned reason. More specifically, the removal time is set in the range from 30 min to 5 hr from the timing when the reaction was started, more preferably from 30 min to 2 hr, and most preferably from 30 min to 1 hr.

Further, corresponding to the vessel used in the reaction, it is possible to remove water contained in the cyanoacetic acid solution beforehand. For example, the method includes the steps of: adding only the cyanoacetic acid aqueous solution and the reaction solvent in the reaction vessel; and then removing water via azeotropic distillation into the outside of the reaction mixture. In this case, it is preferable to remove water via distillation under reduced pressure. Herein, it is preferable to keep the reaction temperature at 80° C. or less, more preferably from 40 to 80° C., and most preferably from 40 to 60° C.

When the compound of the formula (3) is prepared, the order of adding the previously prepared cyanoacetic acid, the alcohol derivative of the formula (2), the solvent and the acid catalyst is not specifically limited. Therefore, corresponding to a type of the reaction vessel used in the preparation, it is possible to change the order of adding the above mentioned materials.

Further, it is possible to variously treat the compound of the formula (3) associated with the application of the compound after the reaction is completed. For example, when a greatly high purity is needed for the compound, it is possible to wash the reaction solution with water, or remove the acid catalyst or the unreacting raw material via filtration, and subsequently to condense the resultant organic layer thereby to recrystallize the resultant product. Alternatively, corresponding to the state of the reaction mixture, it is possible to purify the resultant product via silica gel chromatography.

When the resultant product is recrystallized, a solvent used for the recrystallization is not specifically limited. A preferable solvent from the viewpoint of cost includes acetone, ethyl acetate, toluene, heptane, ethanol, and butanol or the like. Among the solvents, the more preferable solvent includes acetone, ethyl acetate, toluene, and heptane. Those solvents may be used alone or as a mixed solvent.

In an embodiment of the present invention, the purity of the compound represented by the formula (3) is greatly improved through the investigation by the present inventors. Accordingly, it is preferable to use the resultant compound of the formula (3) in the next step without a further purification process after the reaction solution has been washed with water and water has been removed, from the viewpoint of the productivity.

<<Method for Producing Metal Containing Compound>>

Next, a method for preparing a cyanoacetic acid derivative of the following formula (4) will be described.

A method for producing a metal containing compound in an embodiment of the present invention comprises the steps of: undergoing a reaction of the compound of the formula (3) obtained in the above mentioned method with an acid chloride or an acid anhydride thereby to produce the following compound of the formula (4); and subsequently undergoing a reaction of the compound represented by the formula (4) with copper chloride or copper acetate, thereby to produce a metal containing compound of the following formula (5).

[Cyanoacetic Acid Derivative-2 (Cyanoacetic Acid Derivative of Formula (4))

The compound of the formula (3) obtained in an embodiment of the present invention is converted to the cyanoacetic acid derivative of the formula (4) by undergoing a reaction thereof with an acid anhydride or an acid chloride. First, a compound of the formula (4) will be explained hereafter.

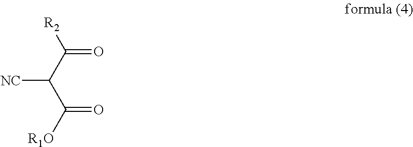

In the formula (4), R₁ is defined the same as R₁ of the formula (3).

Further, in the formula (4), R₂ represents an alkyl group such as a methyl group, an ethyl group, a propyl group, an isopropyl group, a tert-butyl group, a pentyl group, a hexyl group, an octyl group, a dodecyl group, a tridecyl group, a tetradecyl group, a pentadecyl group, a cyclopentyl group, and a cyclohexyl group or the like. Herein, those groups may further have a substituent thereon.

The substituent which may be a substitute on R₂ includes a substituent defined the same as the substituent capable of being positioned on “L” included in the formula (2′). Preferably R2 is an alkyl group having 1 to 4 carbon atoms, more preferably a linear chain structure, more preferably a methyl group or an ethyl group, and most preferably a methyl group.

Here, a method for preparing the compound of the formula (4) is an “acylation” reaction for a so-called active methylene compound. In short, this is a reaction between the active methylene compound and the acid anhydride or the acid chloride. Note the acid anhydride or the acid chloride used in an embodiment of the present invention is not specifically limited.

In the preparation of the compound represented by the formula (4), it is preferable to use a base. Such a base includes sodium methoxide, sodium ethoxide, sodium carbonate, potassium carbonate, sodium hydrogen carbonate, potassium phosphate, potassium hydride, sodium hydride, lithium hydroxide, sodium hydroxide, potassium hydroxide, calcium hydroxide, pyridine, piperidine, piperazine, morpholine, N,N-dimethylaniline, 4-dimethylaminopyridine, triethylamine, DBU and quinoline or the like. Among the above mentioned bases, preferably the base is sodium methoxide, potassium carbonate, pyridine and triethylamine, more preferably sodium methoxide and triethylamine.

A usage rate of the base in the preparation of the compound (4) to the compound of the formula (3) is preferably set in the range from 1.0 eq. to 2.0 eq., more preferably from 1.0 eq. to 1.5 eq., most preferably from 1.0 eq. to 1.2 eq.

In an embodiment of the present invention, a solvent used in the preparation of the compound represented by the formula (4) is not specifically limited as long as the reaction smoothly proceeds in the solvent. Preferably, the solvent is a non-alcohol based material. Such a non-alcohol based solvent includes toluene, xylene, mesitylene, heptane, octane, nonane, ethyl acetate, propyl acetate, ethylene glycol dimethyl ether, tetrahydrofuran, dioxane, acetonitrile, propionitrile, N,N-dimethylformamide, and N,N-dimethylacetamide or the like. Herein, from the viewpoint of cost and collecting feasibility, a more preferable solvent includes toluene and xylene.

A usage rate of the solvent in the preparation of the compound represented by the formula (4) is preferably set in the range from 1-fold to 10-fold, more preferably from 1-fold to 5-fold, most preferably 1-fold to 3-fold, with respect to the compound of the formula (3). In the preparation of the compound represented by the formula (4), an acid anhydride or an acid chloride is used. A usage rate of the acid anhydride or the acid chloride in the preparation of the compound represented by the formula (4) is preferably set in the range from 1.0 eq. to 2.0 eq., more preferably from 1.0 eq, to 1.5 eq., and most preferably from 1.0 eq. to 1.2 eq., with respect to the compound of the formula (3).

{Reaction Process}

A reaction process between the compound represented by the formula (3) and the acid anhydride or the acid chloride is not specifically limited as long as the reaction smoothly proceeds. Preferably, used is either of the following methods for i) preparing a sodium salt of the compound represented by the formula (3) via deprotonation of the active methylene group thereof, or ii) further enhancing the reactivity of the active methylene group in the compound represented by the formula (3) in the presence of a strong organic base.

A reaction temperature in the preparation of the compound represented by the formula (4) is not specifically limited as long as the reaction smoothly proceeds. However, preferably the reaction temperature is set at a temperature as lower as possible in order to suppress a presumed side-reaction such as O-acylation. More specifically, the reaction temperature is preferably set in the range from 30° C. to 100° C., more preferably from 30° C. to 60° C., and most preferably 30° C. to 50° C.

In the preparation of the compound represented by the formula (3), the order of adding the previously prepared compound represented by the formula (3), the solvent, the base, and the acid anhydride or the acid chloride is not specifically limited. Thus, it is possible to change the order of adding the above mentioned reagents corresponding to the type of the reaction vessel used in the reaction. However, preferably the acid anhydride or the acid chloride is lastly added to the reaction mixture. Generally, the above mentioned reactions are exothermic reactions. Therefore, it is more preferable to dropwisely add the acid anhydride or the acid chloride into the reaction mixture.

Further, it is possible to variously treat the compound of the formula (4) after the reaction in association with the application thereof. For example, when significantly high purity is required for the compound, it is possible to remove a resulting salt or an Insoluble substance by washing the reaction solution with water or filtering the reaction solution, and subsequently condense the organic layer thereby to crystallize the resultant material. Alternatively, corresponding to the state of the reaction product, it is possible to purify the product via silica gel chromatography.

When the resultant product is recrystallized, a solvent used for the recrystallization is not specifically limited. Preferably, such a solvent includes acetone, ethyl acetate, toluene, heptane, ethanol and butanol or the like from the viewpoint of cost. Among those solvents, a more preferable solvent includes acetone, ethyl acetate, toluene and heptane. The solvent may be used alone or as a mixed solvent in the combination thereof.

In an embodiment of the present invention, the purity of the compound represented by the formula (4) is greatly improved through the investigation of the present inventors. Accordingly, from the viewpoint of the productivity, it is preferable to use the compound without further purification, after the reaction solution has been washed with water and water has been removed therefrom.

[Metal Containing Compound]

Next, a method for producing a metal containing compound of the formula (5) will be explained in detail.

First, the metal containing compound of the formula (5) will be described in detail.

Here, in the formula (5), R₁ and R₂ are defined the same as the description in the formula (4). The formula (5) may be represented by contributing structures as in the following formulas (5a) and (5b). Here in, the formula (5), the formula (5a) and the formula (5b) essentially represent an aspect of the molecule, the formulas being identical in terms of the resonance hybrid. Note the difference between the bond types described as a covalent bond (shown as “-”: solid line) or a coordinate bond (shown as “- - -”: broken line) is not an essential difference, and does not represent an absolute structural feature.

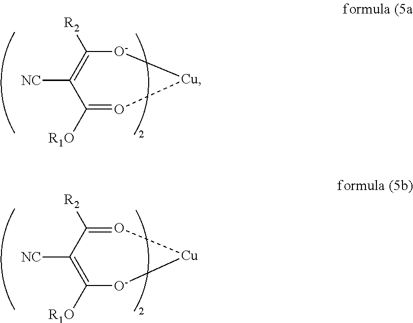

It is preferable to obtain the metal containing compound of the formula (5) via the steps of: preparing the compound of the previously described formula (4); and subsequently undergoing a reaction of the resultant product with a bivalent metal compound. A method for preparing the above mentioned metal containing compound may be conducted following the method as described m an experimental textbook such as “Chelate Chemistry (5), Experimental Methods in Complex Chemistry [I]; ed. by Nankodo Co., Ltd.)

In the preparation of the metal containing compound of the formula (5), a bivalent metal compound used in the preparation includes copper(II) chloride, copper(II) acetate and copper perchlorate or the like. A usage rate of the bivalent metal compound in the preparation of the compound represented by the formula (5) to the compound represented by the formula (4) is preferably set in the range from 0.5 eq. to 0.7 eq., more preferably from 0.5 eq. to 0.6 eq., and most preferably from 0.5 eq. to 0.55 eq.

Here, the solvent used in the preparation of the compound represented by the formula (5) is not specifically limited as long as the reaction smoothly proceeds. However, from the viewpoint of the solubility of the bivalent metal compound used in the preparation, preferably the solvent includes water or an alcohol based solvent. Such an alcohol based solvent includes methanol, ethanol, 1-propanol, 2-propanol, 1-butanol, and 2-butarol or the like. Alternatively, it is possible to use a mixed solvent prepared by adding a non-alcohol based solvent to an alcohol based solvent, if necessary. The non-alcohol based solvent includes toluene, xylene, mesitylene, heptane, octane, nonane, ethyl acetate, propyl acetate, ethylene glycol dimethyl ether, tetrahydrofuran, dioxane, acetonitrile, propionitrile, N,N-dimethylformamide, and N,N-dimethylacetamide or the like.

A usage amount of the solvent in the preparation of the compound represented by the formula (5) is preferably set in the range from 1-fold to 10-fold larger than the amount of the compound represented by the formula (4), and more preferably from 1-fold to 5-fold larger than the amount thereof.

In the preparation of the compound represented by the formula (5), a base may be used. Such a base includes sodium methoxide, sodium ethoxide, sodium carbonate, potassium carbonate, sodium hydrogen carbonate, potassium phosphate, potassium hydride, sodium hydride, lithium hydroxide, sodium hydroxide, potassium hydroxide, calcium hydroxide, pyridine, piperidine, piperazine, morpholine, N,N-dimethylaniline, 4-dimethylaminopyridine, triethylamine, DBU and quinoline or the like. Among the above mentioned bases, preferably the base is sodium methoxide, potassium carbonate, pyridine and triethylamine, more preferably sodium methoxide and triethylamine, and most preferably sodium methoxide.

A usage rate of the base in the preparation of the compound (5) to the compound of the formula (4) is preferably set in the range from 0.6 eq, to 1.0 eq., more preferably from 0.8 eq. to 1.0 eq., and most preferably from 0.9 eq. to 1.0 eq

(Reaction Process)

Process for preparing the compound represented by the formula (5) is not specifically limited as long as the reaction smoothly proceeds. Herein, preferably used is either of the following methods for i) preparing a sodium salt via deprotonation of the active methylene group of the compound represented by the formula (4), or ii) further enhancing the reactivity of the active methylene group in the compound represented by the formula (4) in the presence of a strong organic base.

In an embodiment of the present invention, a reaction temperature in the preparation of the compound represented by the formula (5) is not specifically limited as long as the reaction smoothly proceeds. However, preferably the reaction temperature is set at a temperature as lower as possible in order to suppress a presumed side-reaction such as O-acylation. More specifically, the reaction temperature is preferably set in the range from 10° C. to 100° C., more preferably from 30=0 to 80° C., and most preferably 40° C. to 70° C.

In the preparation of the compound represented by the formula (5), the order of adding the previously prepared compound represented by the formula (4), the solvent, the base, and the bivalent metal containing compound is not specifically limited. Thus, it is possible to change the order of adding the above mentioned reagents corresponding to the type of the reaction vessel used in the reaction. However, preferably the bivalent metal containing compound is lastly added to the reaction mixture. More preferably, the bivalent metal containing compound is dropwisely added into the reaction mixture. Further, it is possible to variously treat the compound of the formula (5) after the reaction in association with the application thereof. For example, when significantly high purity is required for the compound, it is possible to remove a resulting salt or an insoluble substance by washing the reaction solution with water or filtering the reaction solution, and subsequently condense the organic layer thereby to recrystallize the resultant material.

When the resultant product is recrystallized, a solvent used for the recrystallization is not specifically limited. However, preferably such a solvent includes acetone, ethyl acetate, propyl acetate, butyl acetate, acetonitrile, propionitrile, butyronitrile, toluene, heptane, methanol, ethanol, 1-propanol, 2-propanol, 1-butanol, and 2-butanol or the like. The solvent may be used aloe or as a mixed solvent in the combination thereof. When a mixed solvent is used, the content of each solvent may be desirably selected corresponding to the recrystallization efficiency and solubility of the compound represented by the formula (5).

In an embodiment of the present invention, the purity of the compounds represented by the formula (3) and the formula (4) is greatly improved through the investigation by the present inventors. Accordingly, it is preferable to use the resultant compound of the formula (5) without a further purification process in the previous steps (that is, the steps of preparing the cyanoacetic acid derivatives of the formula (3) and the formula (4)). In short, the compound of the formula (5) can be prepared only by conducting the last step (that is, the step of preparing the metal containing compound of the formula (5)) without performing the further purification process thereof. Here, the compound of the formula (5) may have a neutral ligand corresponding to the center metal. Such a representative ligand includes H₂O or NH₂.

Hereinafter, specific examples of the metal containing compound represented by the formula (5) will be listed. However, it should be noted that the embodiment of the present invention is not specifically limited to the examples. Further, the mark “*” shows a connecting position of the representative groups in the following list.

| COMPOUND No. | R₁ | R₂ |
|---|---|---|
| 1 | 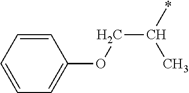 [SMILES: *C(C)COc1ccccc1] | CH₃—* |
| 2 | 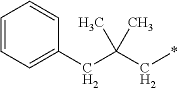 [SMILES: *CC(C)(C)Cc1ccccc1] | CH₃—* |
| 3 | 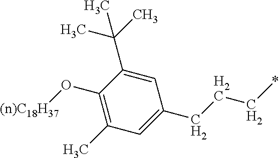 | CH₃—* |
| 4 | 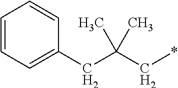 [SMILES: *CC(C)(C)Cc1ccccc1] | CH₃—* |
| 5 | 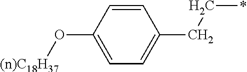 | CH₃—* |
| 6 | 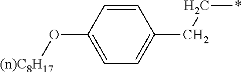 | CH₃—* |
| 7 | 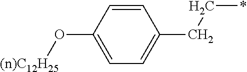 | CH₃—* |
| 8 | 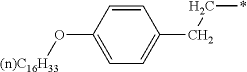 | CH₃—* |
| 9 | 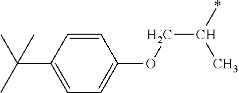 [SMILES: *C(C)COc1ccc(C(C)(C)C)cc1] | CH₃—* |
| 10 | 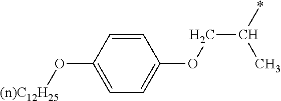 | CH₃—* |
| 11 | 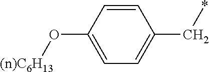 | C₂H₅—* |
| 12 | 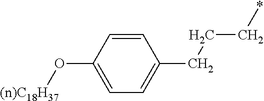 | CH₃—* |
| 13 | 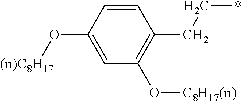 | CH₃—* |
| 14 | 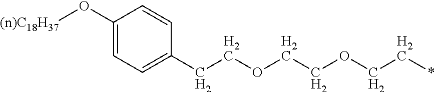 | CH₃—* |
| 15 | 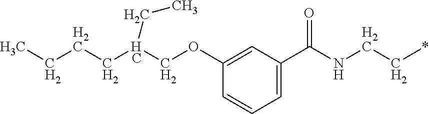 [SMILES: *CCNC(=O)c1cccc(OCC(CC)CCCC)c1] | 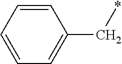 [SMILES: *Cc1ccccc1] |
| 16 | 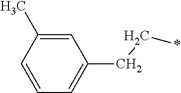 [SMILES: *CCc1cccc(C)c1] | 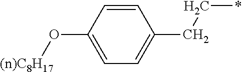 |
| 17 | 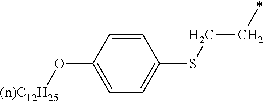 | CH₃—* |
| 18 | 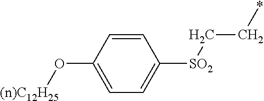 | CH₃—* |
| 19 | 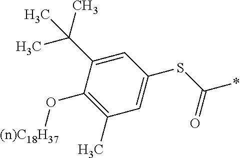 | CH₃—* |
| 20 | 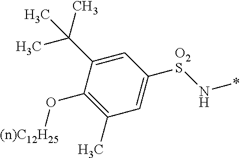 | CH₃—* |
| 21 | 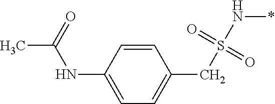 [SMILES: *NS(=O)(=O)Cc1ccc(NC(C)=O)cc1] | CH₃—* |
| 22 | 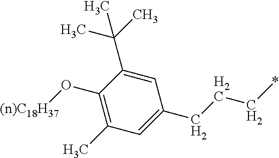 | C₂H₅—* |
| 23 | 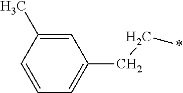 [SMILES: *CCc1cccc(C)c1] | CH₃—* |
| 24 | 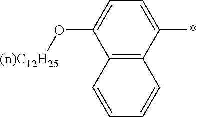 | (n)C₄H₉—* |
| 25 | 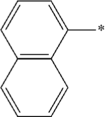 [SMILES: *c1cccc2ccccc12] | 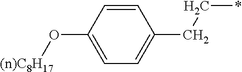 |
| 26 | 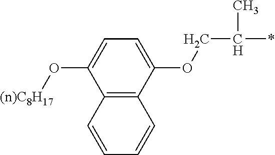 | CH₃—* |
| 27 | 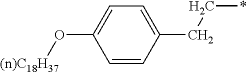 | CF₃—* |

<Application of Metal Containing Compound>

The metal containing compound prepared in a method of the present invention can be used for various applications, especially for the application requiring a copper atom or a copper ion in a complex thereof (for example, resist and copper plating). In such an application, the metal containing compound can be used for a metal supplying source. An example of the application includes a case in which the metal containing compound is added to an electronic photography toner.

When the metal containing compound is used by being added to the electronic photography toner, a dye capable of forming at least one chelate is used in order to form an image. Such a dye capable of forming a chelate may be a material which can chelate with the metal containing compound in an embodiment of the present invention. Those dyes are described in the following patent documents: JPH03-114892, JPH04-62092, JPH04-62094, JPH04-82896, JPH05-16545, JPH05-177958, and JPH05-301470.

Herein, preferably a yellow dye includes a dye of the following formula (6).

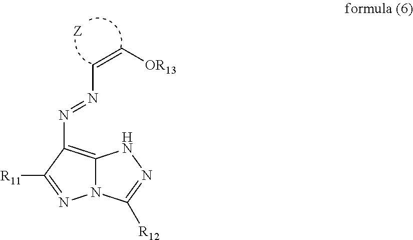

In the above described formula (6), R₁₁ and R₁₂ respectively represent a hydrogen atom or a substituent. R₁₃ represents an alkyl group or an aryl group which may have a substituent thereon. “Z” represents a group of atoms needed for forming a 5-6 membered aromatic ring having 2 carbon atoms.

Preferably, R₁₁ and R₁₂ are substituents, including the same group as the substituent capable of being positioned on “L” of the formula (2′). If said R₁₁ and R₁₂ represent substituents, more preferably said R₁₁ and R₁₂ include an alkyl group, an aryl group or a heteroaryl group. Herein, those groups may further have a substituent, for example, including the same group as the substituent capable of being positioned on “L” of the formula (2′).

R₁₃ represents an alkyl group or an aryl group optionally having a substituent thereon, for example, including capable the same group as the substituent capable of being positioned on “L” of the formula (2′).

an example of the 5-6 membered aromatic ring formed with “Z” and 2 carbon atoms includes a ring system the same as the aryl group and heteroaryl group selected from the substituents capable of being positioned on “L” of the formula (2′). The above mentioned aromatic ring may further have a substituent thereon, and the substituent is prescribed to be the same group as the substituent capable of being positioned on “L” of the formula (2′). Herein, those groups may further have a substituent, for example, including the same group as the substituent capable of being positioned on “L” of the formula (2′).

A dye represented by the formula (6) may be produced referring to the method described in “Chemical Reviews, Vol. 75, 241 (1975)”. That is, the compound of the following formula (A) is diazotized and then undergone via a well-known coupling reaction with the compound of the following formula (B),

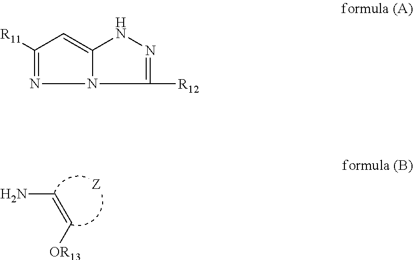

In the above formulae (A) and (B), R₁₁, R₁₂, R₁₃ and “Z” respectively identical to the groups of R₁₁, R₁₂, R₁₃ and “Z” in the formula (6).

Hereinafter, representative examples of a yellow dye of the formula (6) will be shown. However, it should be noted that the present invention is not limited to the specific examples.

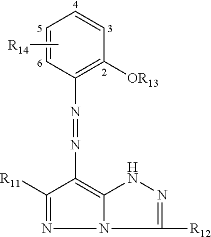

| COMPOUND No. | R₁₁ | R₁₂ | R₁₃ | R₁₄ |
|---|---|---|---|---|
| Y-1 | —CH₃ | —C₄H₉ | —CH₃ | — |
| Y-2 | —C₃H₇(i) | 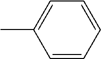 | —CH₃ | — |
| Y-3 | —C₃H₇(i) | —C₂H₅ | —CH₃ | — |
| Y-4 | —CH₃ | —C₂H₅ | —CH₃ | — |
| Y-5 | —C₃H₇(i) | 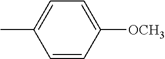 | —CH₃ | 4-Cl |
| Y-6 | —C₃H₇(i) | —C₂H₅ | —CH₃ | 4-CO₂CH₃ |
| Y-7 | —C₃H₇(i) | —C₄H₉ | —CH₃ | 5-CO₂CH₃ |
| Y-8 | —C₄H₉(t) | —C₄H₉ | —CH₃ | — |
| Y-9 | —C₃H₇(i) | 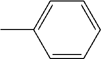 | —C₃H₇(i) | — |
| Y-10 | —C₃H₇(i) | 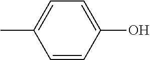 | —CH₃ | — |
| Y-11 | —C₃H₇(i) | —C₃H₇ | —CH₃ | 5-Cl |
| Y-12 | —C₃H₇(i) | 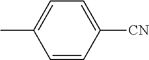 | —CH₃ | — |
| Y-13 | —C₄H₉(t) | 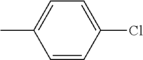 | —CH₃ | — |
| Y-14 | —SCH₃ | 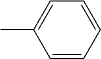 | —CH₃ | — |
| Y-15 | 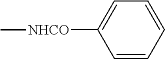 | —C₂H₅ | —CH₃ | — |
| Y-16 | 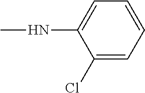 | —C₂H₅ | —CH₃ | — |
| Y-17 | —OCH₃ | —C₄H₉ | —CH₃ | — |
| Y-18 | —C₄H₉(t) | —C₄H₉ | —CH₃ | 4-CO₂H |
| Y-19 | —C₃H₇(i) | 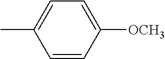 | —CH₃ | — |
| Y-20 | —C₃H₇(i) | 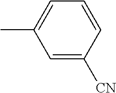 | —CH₃ | — |
| Y-24 | —C₃H₇(i) | —C₂H₅ | —CH₃ | 5-Cl |
| Y-25 | —C₄H₉(t) | —C₄H₉ | —CH₃ | 5-Cl |
| Y-26 | —C₄H₉(t) | —C₂H₅ | —CH₃ | 5-Cl |
| Y-27 | —C₄H₉(t) | 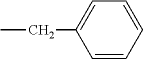 | —CH₃ | 5-Cl |
| Y-28 | —C₄H₉(t) | 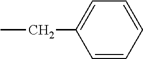 | —CH₃ | — |
| Y-29 | —C₄H₉(t) | 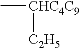 | —CH₃ | 5-Cl |
| Y-30 | —C₄H₉(t) | —C₆H₁₃ | —CH₃ | 5-Cl |
| Y-31 | —C₄H₉(t) | —CH₃ | —CH₃ | 5-Cl |
| Y-32 | —C₄H₉(t) | —CH₃ | —CH₃ | — |

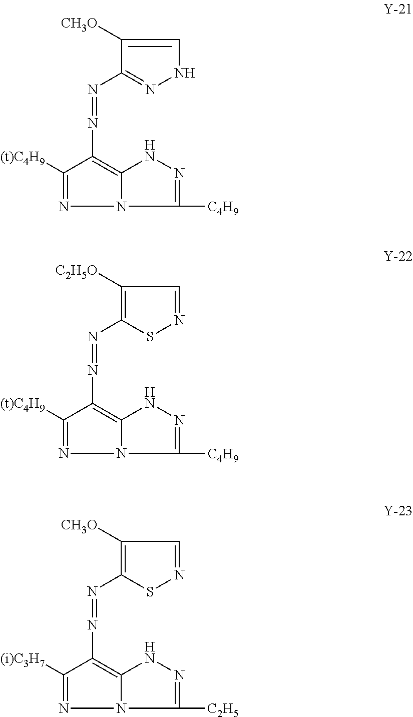

Preferably a magenta dye includes the material of the following formula (7)

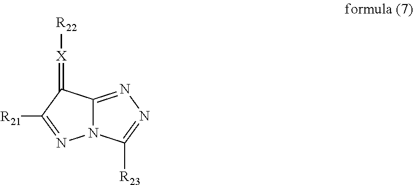

In the above formula (7), R₂₁ represents a hydrogen atom, a halogen atom or a substituent and R22 represents an aryl group or a heteroaryl group which may have a substituent thereon. Further, “X” represents a methine group or a nitrogen atom.

Further, R₂₃ represents a structure of the following formula (8) or (9).

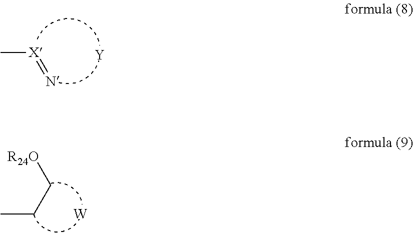

In the above formulae (8) and (9), “X′” represents a carbon atom or a nitrogen atom, “Y” represents a group of atoms which form a nitrogen containing aromatic heterocycle. Further, “W” represents a group of atoms which form an aryl group or a heteroaryl group. R₂₄ represents an alkyl group.

Preferably, R₂₁ is a substituent, including a group the same as the substituent capable of being positioned on “L” in the formula (2′). If said R₂₁ represents a substituent, more preferably R₂₁ includes an alkyl group, an aryl group or a heteroaryl group. Those groups may further have another substituent, for example, including a group the same as the substituent capable of being positioned on “L” of the formula (2′).

R₂₂ represents an aryl group or a heteroaryl group which may have a substituent thereon, for example, including a group the same as an aryl group or a heteroaryl group which is capable of being positioned thereof. Those groups may further have another substituent, for example, including a group the same as the substituent capable of being positioned on “L” of the formula (2′).

“Y” represents a group of atoms which form a nitrogen containing aromatic heterocycle, for example, including a group corresponding to a group within the heteroaryl groups selected from a group the same as the substituent capable of being positioned on “L” of the formula (2′).

“W” represents a group of atoms which form an aryl group or a heteroaryl group. An example of the aryl group or a heteroaryl group thus formed includes a group the same as the aryl group or the heteroaryl group selected from a group the same as the substituent capable of being positioned on “L” of the formula (2′).

A dye of the formula (7) can be prepared following a well-known method. For example, the azomethine chromophore in the formula (7) can be prepared following the oxidation coupling method as described in the patent documents: JPS63-113077, JFH03-275767, and JPH04-89287.

Next, specific examples of the magenta dye represented by the formula (7) will be shown. Herein, it should be noted that the present invention is not limited to such examples.

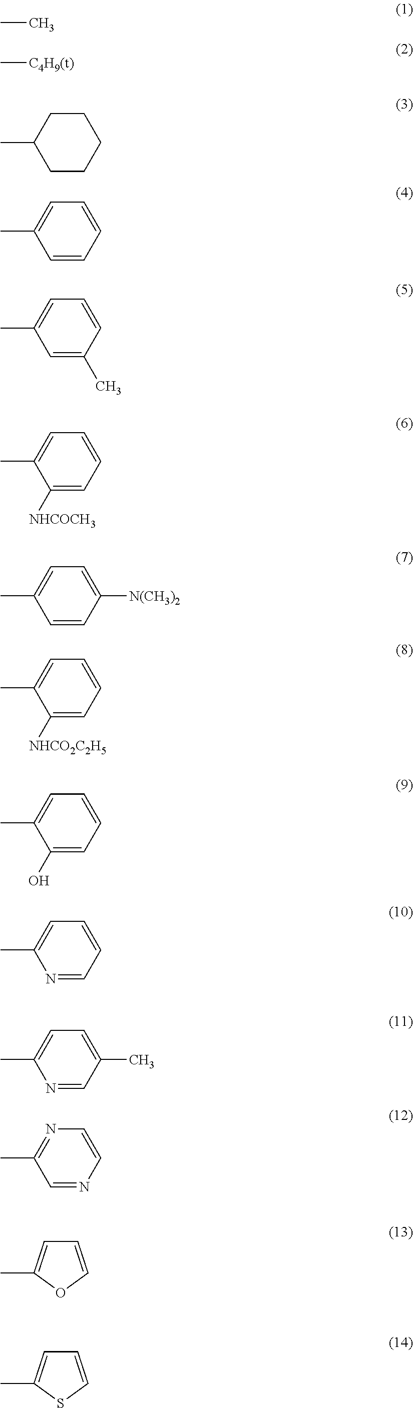

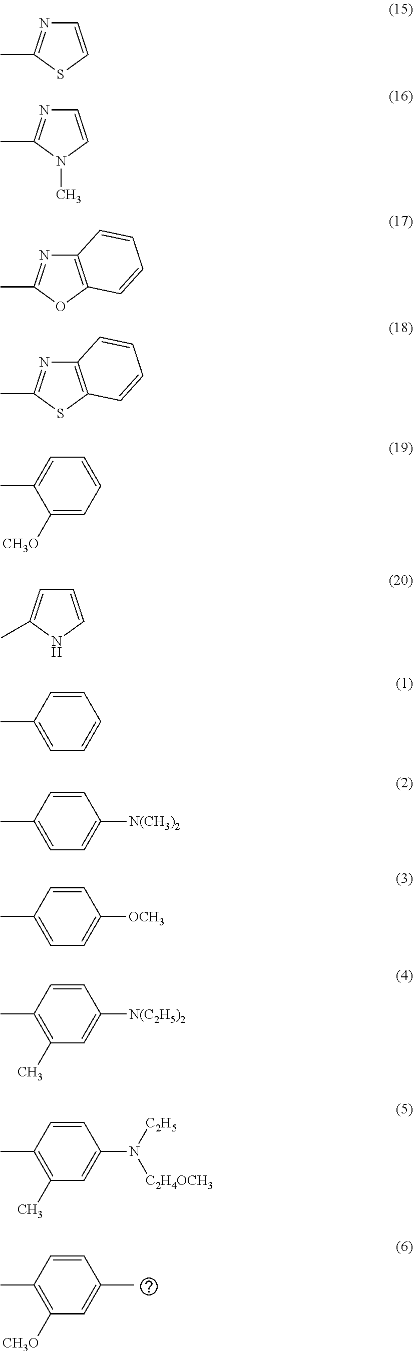

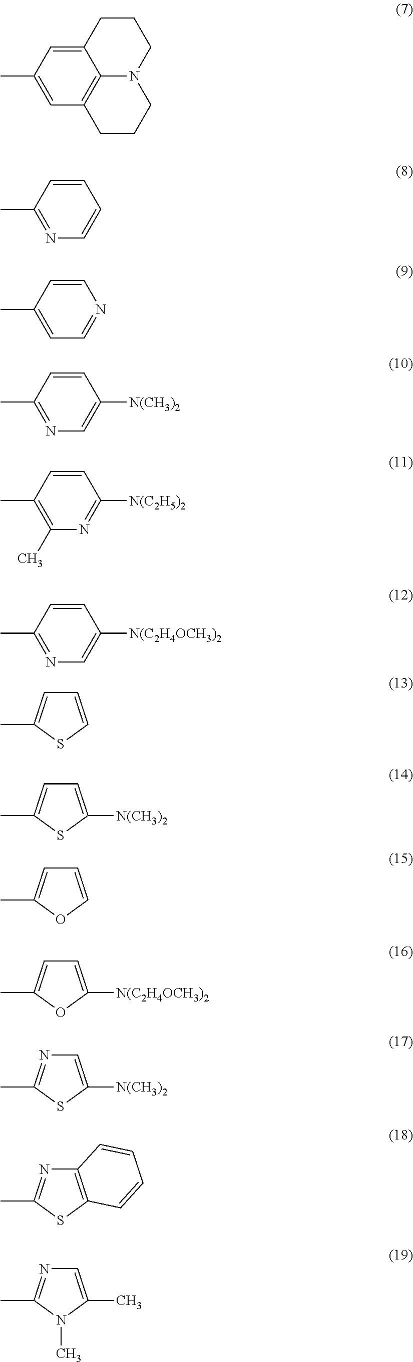

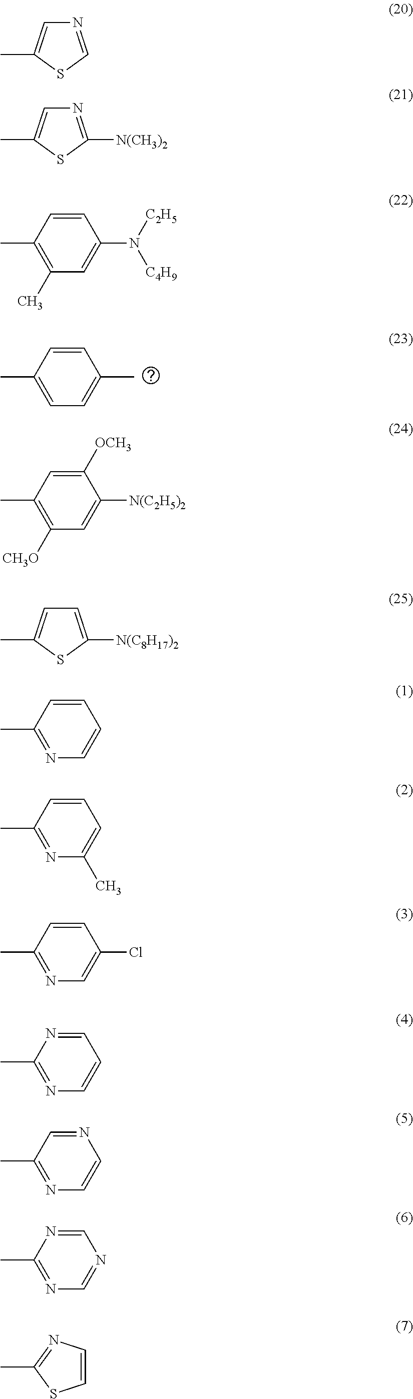

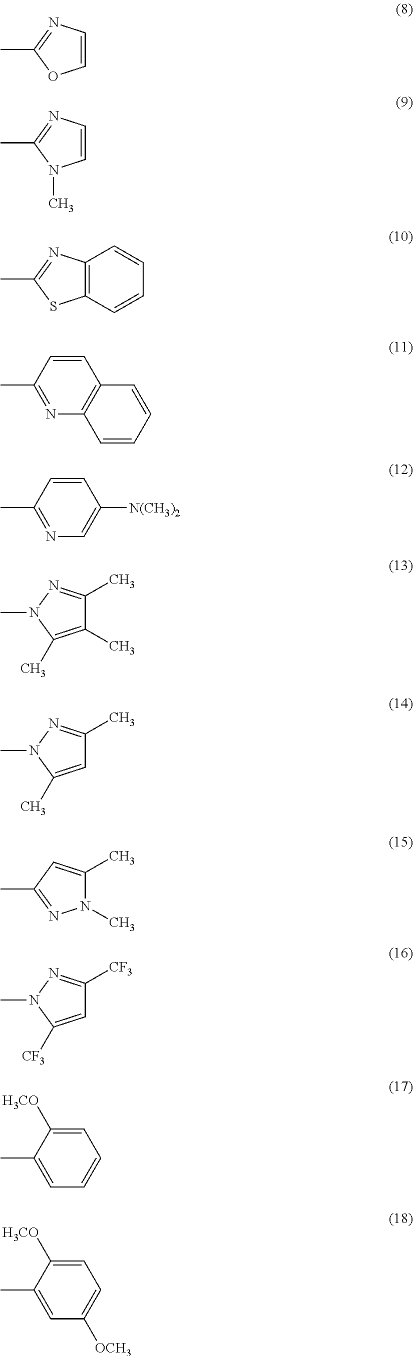

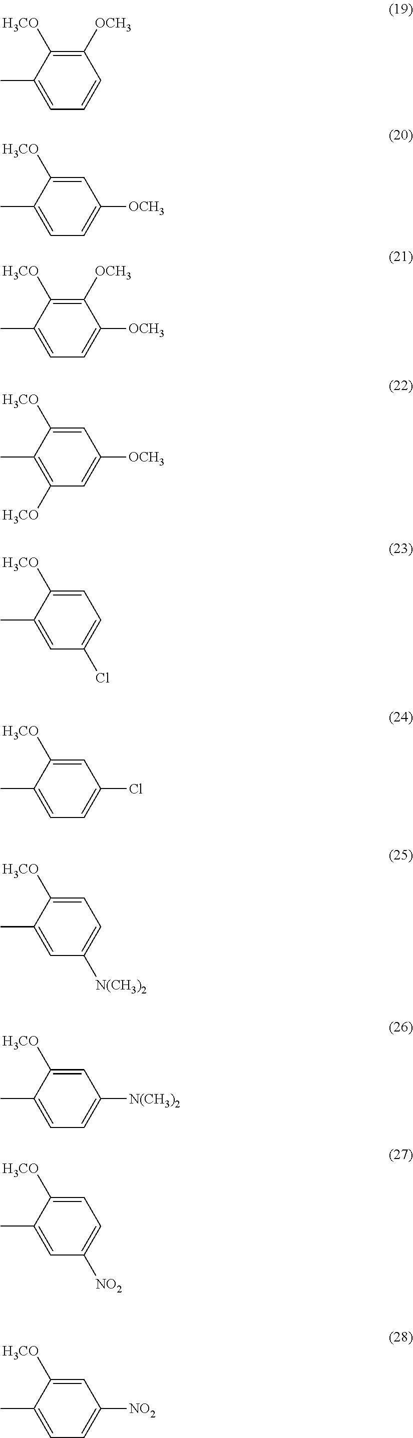

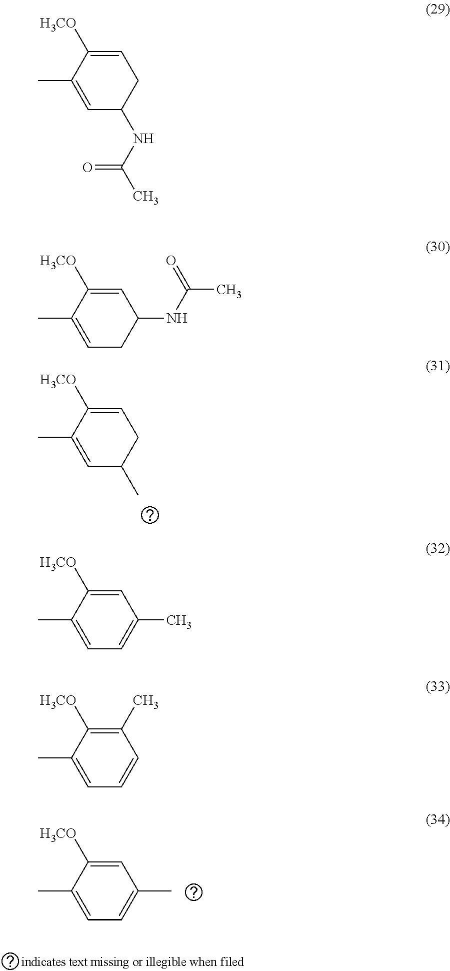

| DYE | R₂₁ | R₂₂ | R₂₃ | X |
|---|---|---|---|---|
| M-1 | (1) | (2) | (15) | N |
| M-2 | (1) | (6) | (9) | N |
| M-3 | (1) | (6) | (10) | N |
| M-4 | (1) | (11) | (7) | N |
| M-5 | (1) | (11) | (8) | N |
| M-6 | (1) | (17) | (8) | CH |
| M-7 | (1) | (20) | (6) | CH |
| M-8 | (1) | (21) | (7) | CH |
| M-9 | (2) | (4) | (3) | N |
| M-10 | (2) | (4) | (5) | N |
| M-11 | (2) | (4) | (6) | N |
| M-12 | (2) | (8) | (3) | CH |
| M-13 | (2) | (10) | (4) | CH |
| M-14 | (2) | (11) | (1) | N |
| M-15 | (2) | (13) | (15) | CH |
| M-16 | (2) | (14) | (1) | CH |
| M-17 | (2) | (14) | (4) | N |
| M-18 | (2) | (19) | (5) | CH |
| M-19 | (3) | (5) | (2) | N |
| M-20 | (3) | (16) | (9) | CH |
| M-21 | (3) | (18) | (10) | CH |
| M-22 | (4) | (3) | (2) | CH |
| M-23 | (4) | (3) | (14) | N |
| M-24 | (4) | (7) | (13) | N |
| M-25 | (4) | (10) | (11) | N |
| M-26 | (4) | (13) | (12) | CH |
| M-27 | (4) | (15) | (11) | CH |
| M-28 | (5) | (9) | (14) | CH |
| M-29 | (5) | (12) | (13) | CH |
| M-30 | (5) | (21) | (12) | N |
| M-31 | (10) | (2) | (15) | N |
| M-32 | (16) | (13) | (15) | CH |
| M-33 | (17) | (18) | (15) | N |
| M-34 | (18) | (21) | (15) | CH |
| M-35 | H | (7) | (16) | CH |
| M-36 | H | (16) | (16) | N |
| M-37 | (2) | (4) | (5) | CH |
| M-38 | (2) | (22) | (5) | CH |
| M-39 | (2) | (25) | (17) | CH |
| M-40 | (1) | (25) | (17) | CH |
| M-41 | (4) | (25) | (17) | CH |
| M-42 | (4) | (22) | (17) | CH |
| M-43 | (4) | (22) | (28) | CH |
| M-44 | (2) | (14) | (18) | CH |
| M-45 | (2) | (25) | (25) | CH |

Preferably, a cyan dye includes a compound of the following formula (10).

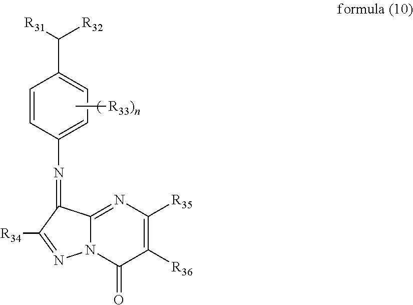

In the formula (10), R₃₁ and R₃₂ respectively represent a substituted alkyl group or an unsubstituted alkyl group, and R₃₂ represents a substituent. The term of “n” represents an integer from 0-4. Herein, if “n” represents 2 or more, a plurality of the substituents represented by may be the same or the different substituents. Each of the groups of R₃₄, R₃₅ and R₃₆ represents an alkyl group. Herein, R₃₄, R₃₅ and R₃₆ may be the same groups or the different groups. Note R₃₅ and R₃₆ are alkyl groups having 3-8 carbon atoms.

A substituent of R₃₃ includes a group the same as the substituent capable of being positioned on “L” of the formula (2′).

A dye represented by the formula (10) may be prepared by following the well-known method. For example, the dye of the formula (10) may be prepared by following the oxidation coupling methods as described in JP2000-255171, JP2001-334755, and JP2002-234266.

Hereinafter, specific examples of the cyan dye represented by the formula (10) will be described in detail. Herein, it should be noted that the present invention is not limited to such specific examples.

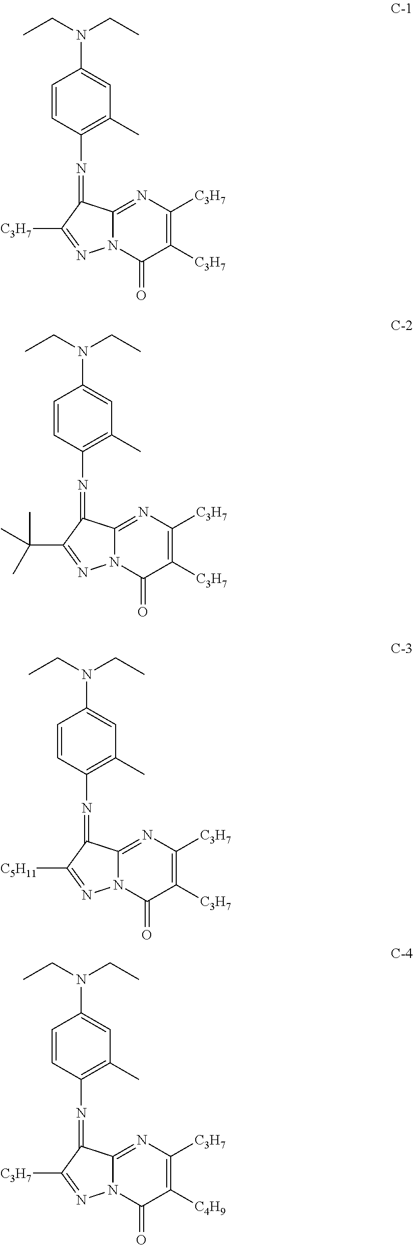

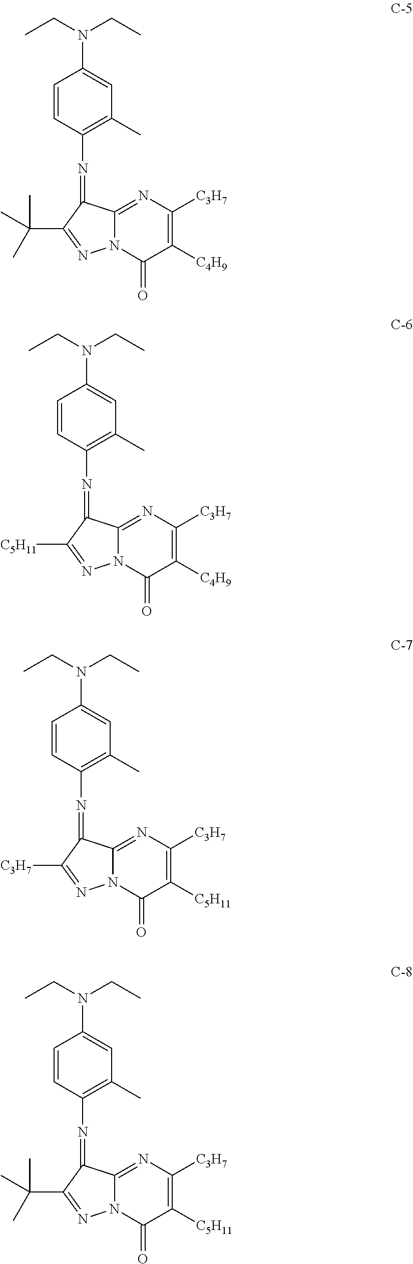

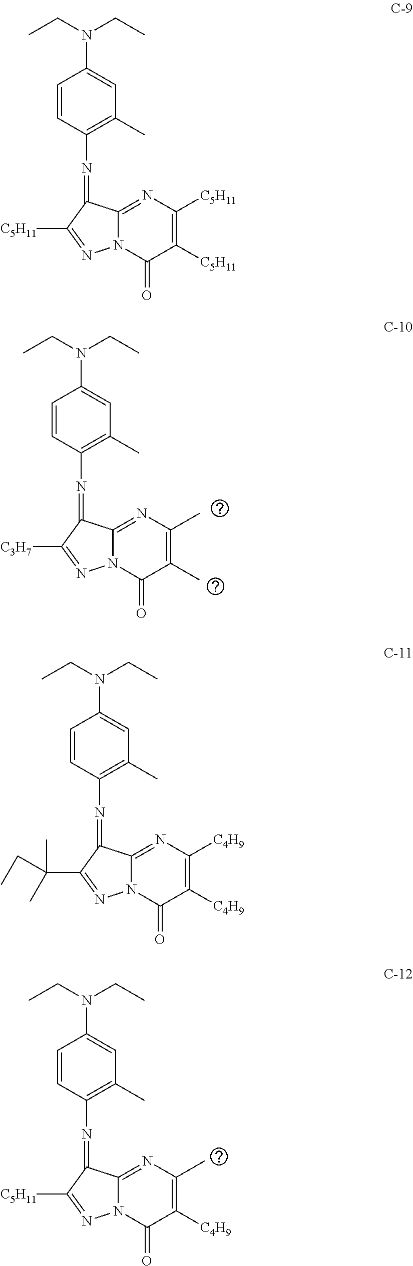

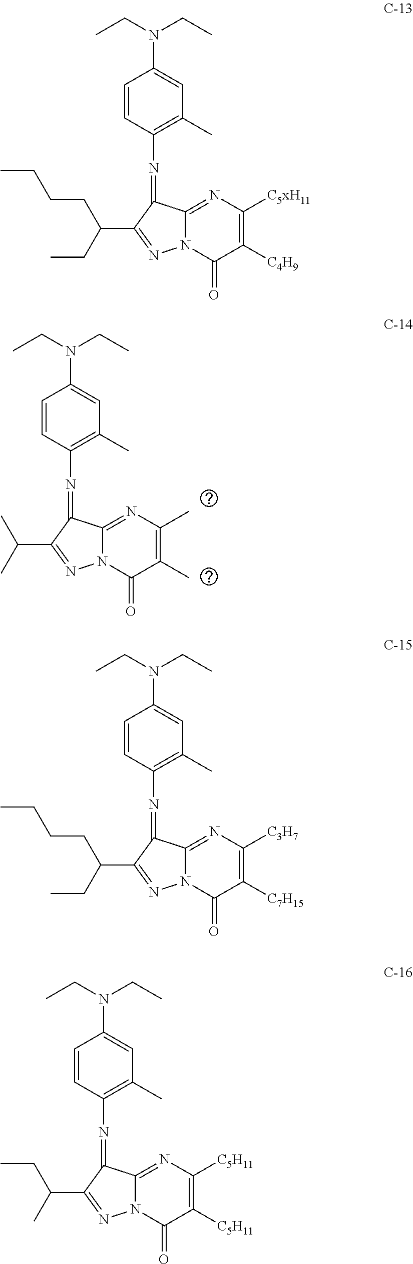

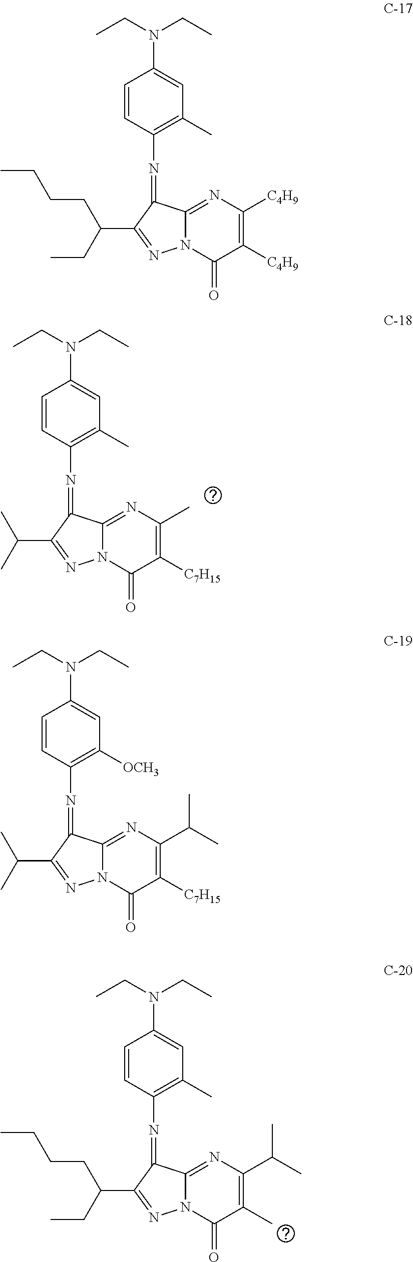

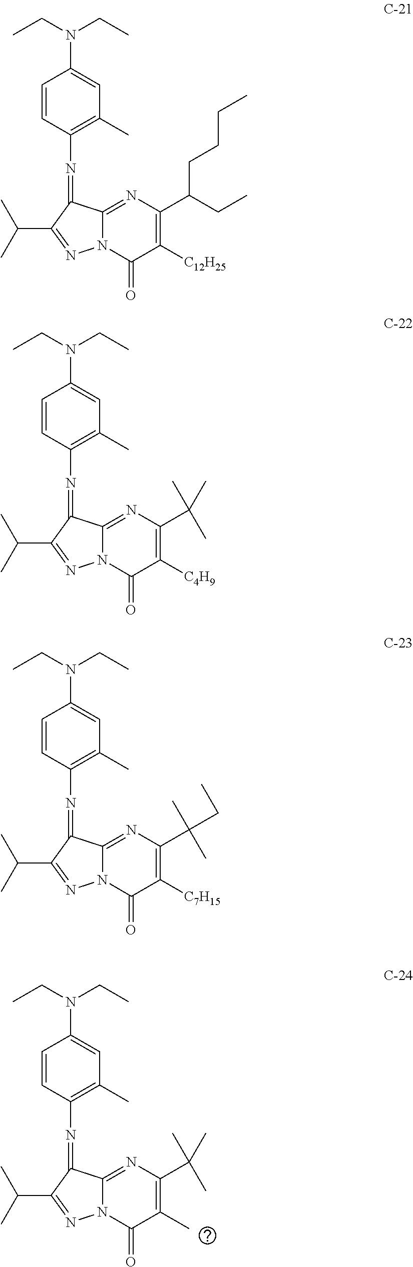

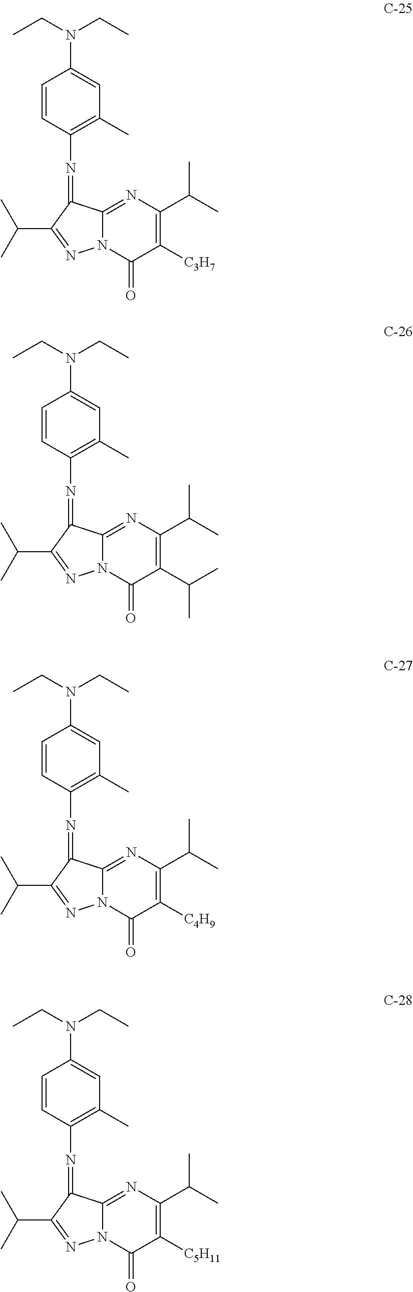

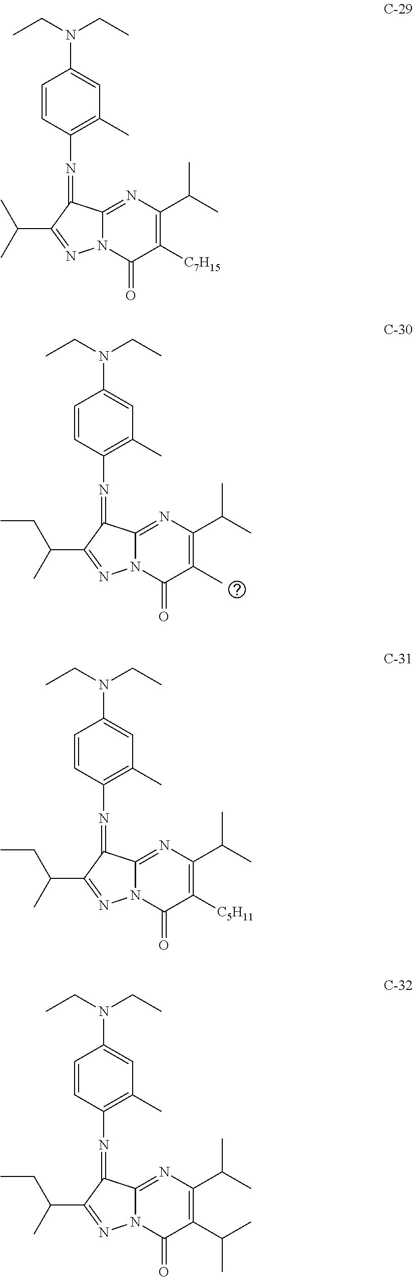

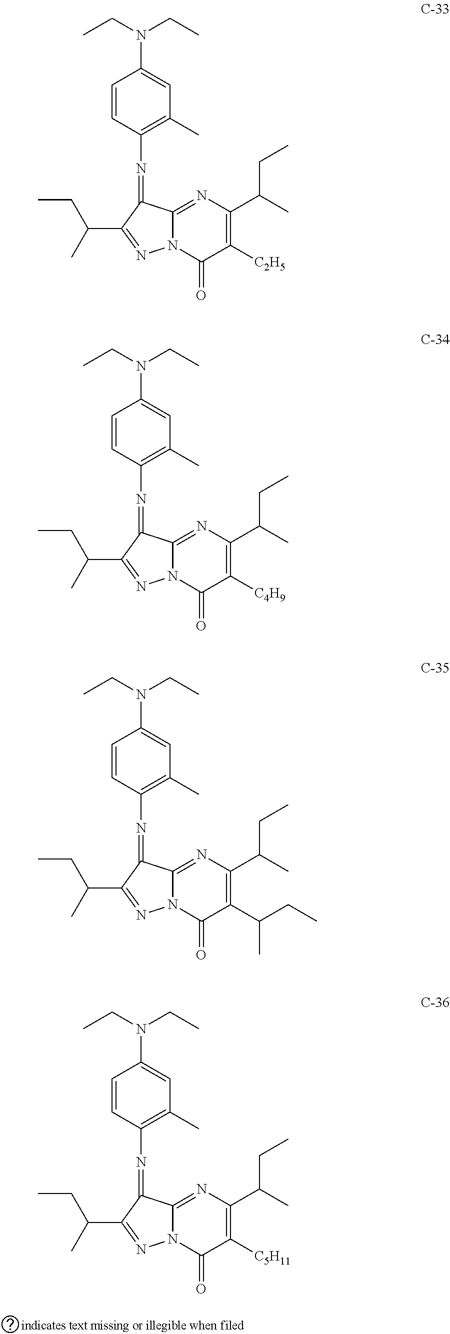

The metal containing compound of the formula (5) obtained by the method in an embodiment of the present invention may form a metal chelating dye represented by the following formula (11), (12) or (13) with the dye of the formula (6), (7) or (10), respectively.

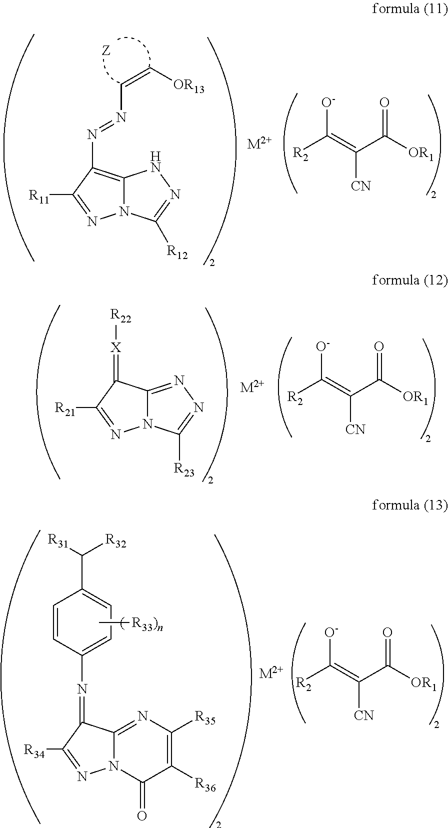

In the formulas (11)-(13), R₁₁, R₁₂, R₁₃, R₂₁, R₂₂, R₂₃, R₃₁, R₃₂, R₃₃, R₃₄, R₃₅ and R₃₆ respectively represent groups the same as the substituents defined in the above mentioned formulae (6), (7) and (10). Further, R₁ and R₂ respectively represent groups the same as the substituents defined in the above mentioned formula (5), and M²⁺ represents a bivalent copper ion.

Those chelating dyes can be used for various applications besides an electronic photography toner. The chelating dyes can be applied to the toners by following the methods as described in the patent documents: JPH10-265690, JP2000-345059 and WO2011/010509A1. However, those applications and usage methods of the dyes in an embodiment of the present invention are not limited only to the above mentioned methods.

Hereinafter, the electronic photography toner using the compound of the formula (5) in an embodiment of the present invention will be described in detail.

(Method for Dispersing Dye)

It is possible to produce the electronic photography toner by the methods comprising the steps of: directly dispersing a dispersion liquid of a dye in a binder resin or mixing a dispersion liquid of coloring micro particles in a binder resin; and preparing the toners in a kneading/grinding method, a suspension polymerization method, an emulsion polymerization method, an emulsification/dispersion granulation method, a capsulation method and other well-known methods. Among those production methods, it is preferable to use an emulsion polymerization method from the viewpoint of cost and stability in the preparation, when further taking it into consideration that the particle size of the toners is made smaller corresponding to the image more highly visualized. The emulsion polymerization method for producing the toner particles comprises the steps of: mixing a thermoplastic resin emersion produced via emulsion polymerization with a dispersion liquid including the components of the toner particle such as dispersion substances of other solid dye particles; slowly agglomerating the particles by balancing the repulsion force and the agglomeration force on the surfaces of the micro particles produced via controlling the pH value of the liquid mixture; and gathering the produced micro particles via controlling the particle size/the particle size distribution with simultaneously heating and stirring the liquid mixture, thereby to control the fusion between the micro particles/the shaping thereof.

When the dye dispersion liquid is directly dispersed, it is possible to perform the dispersion process by using a beads disperser, a high speed agitation disperser, and a medium type agitator or the like. Herein, it is also possible to disperse a dispersion liquid in the same manner as described in the preparation of the following coloring micro particle dispersion substance. That is, such a method comprises the steps of: dissolving (or dispersing) the dye in an organic solvent; dispersing as emulsification in water; and removing the organic solvent.

(Coloring Micro Particle)

In an aspect of the electronic photography toner in an embodiment of the present invention, it is possible to at least disperse coloring micro particles in a thermoplastic resin. Herein, the coloring micro particle includes at least the metal complex compound of the formula (5). The dispersion particle size of the coloring micro particle can be controlled by a dispersing method such as a liquid drying method described hereinafter.

Further, it is preferable to produce the electronic photography toner by further adding a resin having a different composition from the above mentioned thermoplastic resin or a solvent having a high boiling point to the toner. Moreover, it is also possible to disperse the coloring micro particle (or a dispersion liquid in which the dye is only dispersed) in the thermoplastic resin, instead of directly dispersing the dye into a generally known binder resin for toner in which the above mentioned dye is used.

Note the dye in the coloring micro particle is dissolved in the resin at a molecular level. This allows a component such as a covering particle which blocks light to be omitted in the toner. Hereby, this effect results in the improvement of the transparency for a single color derived from the corresponding toner, and further in the improvement of the transparency for overlaid colors derived from the corresponding toners.

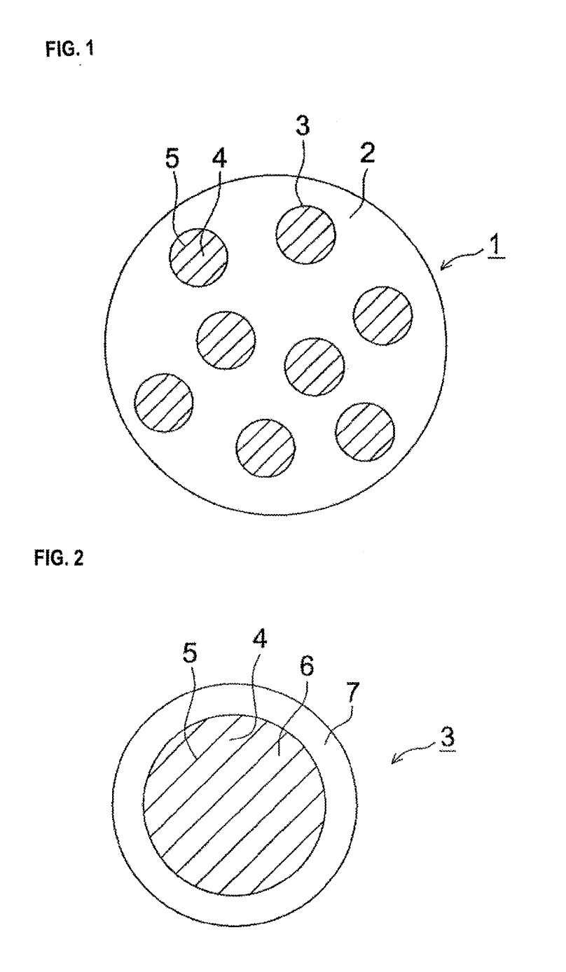

Here, FIG. 1 schematically shows a cross-sectional diagram of the toner particle for electronic photograph 1 in an embodiment of the present invention. Herein, the toner particle is produced by dispersing the coloring micro particle 3 in the thermoplastic resin 2. Further, in an example of a preferable embodiment, as shown in FIG. 2, the coloring micro particle 3 may be covered by a shell resin (or shell) 7. In such a case, the combination of a resin A composing an inside part (or core) 6 of the coloring micro particle 3 and the thermoplastic resin (or binder resin) 2 is not limited to a specific way, allowing a selection range of the materials thereof to be greatly increased. Further, if the shell resin (or shell) 7 is made of the same material with respect to the 4 cored toners (that is, yellow, magenta, cyan and black), it is possible to produce the 4 different toners under the same production conditions. This results in the great advantage for the cost. Moreover, a dye (or oil-soluble dye) 5 of a coloring agent does not migrate from the coloring micro particle 3 to the outside thereof (that is, the dye is not exposed on the surface of the coloring micro particle 3). Accordingly, this may prevent the dye form subliming or contaminating oil at a time of the heat fixing, which is generally considered as a drawback of the toner using a dye.

(Method for Preparing Coloring Micro Particles)

Next, an example of a method for preparing coloring micro particles in a preferable embodiment of the present invention will be described.

Coloring micro particles in an embodiment of the present invention can be obtained by a method, for example, comprising the steps of: dissolving (or dispersing) a dye (or material containing a dye and a resin, an organic solvent with a high boiling point, and an additive) in an organic solvent; emulsifying and dispersing the dye in water; and removing the organic solvent (or called a liquid drying method). Further, if the coloring micro particles are prepared by covering a shell resin (or shell) on the particles, the method comprises the steps of: adding a monomer having a polymerizable unsaturated double bond to the coloring micro particles; conducting the emulsion polymerization in the presence of a polymerization agent; depositing the produced resin on the surface of the cores in parallel with the polymerization; and producing the coloring micro particles each of which has a core shell structure.

Alternatively, for example, the method comprises the steps of; forming an aqueous dispersion material of resin micro particles in advance via conducting the emulsion polymerization; mixing an organic solution prepared by dissolving a dye in the resulting aqueous dispersion material of resin micro particles; subsequently impregnating the dye into the resin micro particles; and forming a shell on the core surface of each resultant coloring micro particle. As mentioned above, various methods can be used for preparing the coloring micro particles each of which has a shell.

Preferably the shell is made of an organic resin. Herein, there is a method for forming the shell, for example, comprising the steps of: gradually dropping a resin solution prepared by dissolving the resin in an organic solvent; and having the separated resin adhere to the core surface of each coloring micro particle at the same timing of the separation.

However, in an embodiment of the present invention, preferably a method for forming the shell comprises the steps of: forming coloring micro particles each of which becomes a core containing a dye and a resin; subsequently adding a monomer having a polymerizable unsaturated double bond; conducting the emulsion polymerization in the presence of a polymerization agent; and having the resultant resin adhere to the surface of each core thereby to form a shell on the core.

Besides the above mentioned methods, a shell may be formed by dispersing a dye in water with a surfactant via using a bead disperser, a high speed agitation disperser, and medium type agitator or the like.

(General Surfactant)

When the coloring micro particles in a preferable embodiment of the present invention are prepared via the emulsification, it is possible to use a general anion based emulsifier (or surfactant), and/or a non-ion based emulsifier (or surfactant) may be used if necessary.

The above mentioned general non-ion based emulsifier includes, for example, polyoxyethylene alkyl ethers such as polyoxyethylene lauryl ether and polyoxyethylene stearyl ether or the like; polyoxyethylene alkylphenyl ethers such as polyoxyethylene nonylphenyl ether or the like; sorbitan higher fatty acid esters such as sorbitan monolaurate, sorbitan monostearate, and sorbitan trioleate or the like; polyoxyethylene sorbitan higher fatty acid esters such as polyoxyethylene sorbitan monolaurate; polyoxyethylene higher fatty acid esters such as polyoxyethylene monolaurate and polyoxyethylene monostearate or the like; glycerin higher fatty acid esters such as oleic acid monoglyceride and stearic acid monoglyceride or the like; and polyoxyethylene-polyoxypropylene-block copolymer or the like.

Further, the above mentioned anion based emulsifier includes, for example, higher fatty acid salts such as sodium oleate; alkylarylsulfonic acid salts such as sodium dodecylbenzenesulfonate; alkyl sulfonic acid esters such as sodium lauryl sulfate; polyoxyethylene alkyl ether sulfonic acid salts such as sodium polyoxyethylene lauryl ether sulfate; polyoxyethylene alkyl aryl ether sulfonic acid salts such as sodium polyoxyethylene nonylphenyl ether sulfate; and alkyl sulfosuccinic acid salts such as sodium monooctyl sulfosuccinate, sodium dioctyl sulfosuccinate and sodium polyoxyethylene lauryl sulfosuccinate, and the derivatives thereof.

(Dye)

Hereinafter, a dye used in an embodiment of the present invention will be described in detail.

In an embodiment of the present invention, it is possible to use a generally known dye. Preferably the dye is an oil-soluble dye in an embodiment of the present invention. Such an oil-soluble dye is generally soluble in an organic solvent having no water-soluble group such as a carboxyl group and a sulfonic group, while this type of dye is insoluble in water. Herein, it should be noted that the oil-soluble dye may include such a dye as prepared via salt formation with a long-chain basic group, resulting in the increase in the oil solubility. For example, a dye prepared by salt formation is known, which is made via salt formation of an acidic dye, a direct dye or a reactive dye with a long-chain amine.

Hereinafter, examples of the above mentioned dye include, for example, Orient Chemical Industries Co., Ltd.: Valifast Yellow 4120, Valifast Yellow 3150, Valifast Yellow 3108, Valifast Yellow 2310N, Valifast Yellow 1101, Valifast Red 3320, Valifast Red 3304, Valifast Red 1306, Valifast Blue 2610, Valifast Blue 2606, Valifast Blue 1603, Valifast Blue 2610, Oil Yellow GG-S, Oil Yellow 3G, Oil Yellow 129, Oil Yellow 107, Oil Yellow 105, Oil Scarlet 308, Oil Red RR, Oil Red OG, Oil Red 5B, Oil Pink 312, Oil Blue BOS, Oil Blue 613, Oil Blue 2N, Oil Black BY, Oil Black BS, Oil Black 860, Oil Black 5970, Oil Black 5906, Oil Black 5905; Nippon Kayaku Co., Ltd.: Kayaset Yellow SF-G, Kayaset Yellow K-CL, Kayaset Yellow GN, Kayaset Yellow A-G, Kayaset Yellow 2G, Kayaset Red 5F-4G, Kayaset Red K-BL, Kayaset Red A-BR, Kayaset Magenta 312, Kayaset Blue K-FL; Arimoto Chemical Co., Ltd.: FS Yellow 1015, FS Magenta 1404, FS Cyan 1522, FS Blue 1504, C. I. Solvent Yellow 88, 83, 82, 79, 56, 29, 19, 16, 14, 04, 03, 02, 01, C. I. Solvent Red 84:1, C. I. Solvent Red 84, 218, 132, 73, 72, 51, 43, 27, 24, 18, 01, C. I. Solvent Blue 70, 67, 44, 40, 35, 11, 02, 01, C. I. Solvent Black 43, 70, 34, 29, 27, 22, 7, 3, C. Z. Solvent Violet 3, C. I. Solvent Green 3 and 7, Plast Yellow DY 352, Plast Red 8375; Mitsui Chemicals, Inc.: MS Yellow HD-180, MS Red G, MS Magenta HM-1450H, MS Blue HM-1384; Sumitomo Chemical Co., Ltd.: ES Red 3001, ES Red 3002, ES Red 3003, TS Red 305, ES Yellow 1001, ES Yellow 1002, TS Yellow 118, ES Orange 2001, ES Blue 6001, TS Turq Blue 618; and Bayer AG: MACROLEX Yellow 6G, Ceres Elue GNNEOPAN Yellow 075, Ceres Blue GN, and MACROLEX Red Violet R or the like. Herein, it should be noted that the dyes in an embodiment of the present invention are not limited to the above examples.

Further, a dye dispersion can be used as an oil-soluble dye including, for example, C. I. Disperse Yellow 5, 42, 54, 64, 79, 82, 83, 93, 99, 100, 119, 122, 124, 126, 160, 184:1, 186, 198, 199, 204, 224 and 237; C. I. Disperse Orange 13, 29, 31:1, 33, 49, 54, 55, 66, 73, 118, 119 and 163; C. I. Disperse Red 54, 60, 72, 73, 86, 88, 88, 91, 92, 93, 111, 126, 127, 134, 135, 143, 145, 152, 153, 154, 159, 164, 167:1, 177, 181, 204, 206, 207, 221, 239, 240, 258, 277, 278, 283, 311, 323, 343, 348, 356 and 362; C. I. Disperse Violet 33; C. I. Disperse Blue 56, 60, 73, 87, 113, 128, 143, 148, 154, 158, 165, 165:1, 165:2, 176, 183, 185, 197, 198, 201, 214, 224, 225, 257, 266, 267, 287, 354, 358, 365 and 368; and C. I. Disperse Green 6:1 and 9 or the like.

Moreover, most preferable examples of an oil-soluble dye besides the above examples include phenols and naphthols; cyclic methylene compounds such as pyrazolone and pyrazolotriazole; azomethine dyes derivatized from a color coupler like an open-chain methylene compound; and an indoaniline dye or the like.

Such dyes are preferably described in patent documents, for example, JPH03-114892, JPH04-62092, JPH04-62094, JPH04-82896, JPH05-16545, JPH05-177958, and JPH05-301470.

(Particle Size)

A volume average particle size of the coloring micro particle in an embodiment of the present invention is preferably set in the range from 10 nm to 10 μm. If the volume average particle size of the coloring micro particle is set smaller than 10 nm, the surface area per unit volume becomes larger. This deteriorates the covering effect of the polymer in the coloring micro particle to wrap up the dye, likely resulting in the instability of the coloring micro particle, thereby to deteriorate the storage stability of the particle.

In contrast, if the volume average particle size of the coloring micro particle is set larger than 1 μm, such micro particles tend to precipitate when the micro particles are prepared, resulting in the deterioration of the stagnation stability of the particle. Further, when a toner is prepared using such large micro particles, the resultant toner is likely to markedly deteriorate the lustrous appearance as well as the transparent appearance.

Accordingly, preferably the volume average particle size of the coloring micro particle is in the range from 10-1 μm, more preferable form 10-500 nm, and most preferably from 10-100 ran.

The volume average particle size of the micro particle may be measured by a dynamic light scattering method, a laser diffraction method, a centrifugation method, an FFF method, and an electric detection method. In an embodiment of the present invention, preferably the volume average particle size of the micro particle is measured by a dynamic light scattering method using Malvern Zetasizer (Malyern Instruments Ltd.).

(Content of Dye)

The coloring micro particle in an embodiment of the present invention preferably has a dye content as in the range from 10 to 70 mass %. The dye content in the range from 10 to 70 mass % allows the coloring micro particle to have a sufficient concentration of the dye, thereby to exert the protection ability of the resin for the coloring material together with the excellent storage stability of the micro particle dispersion. This can prevent the particle from being agglomerated, thereby to suppress the increase in the agglomerated particle size.

(Content of Metal Containing Compound)

The metal containing compound of the formula (5) may be used alone or in combination of two different compounds. Herein, the content of the metal containing compound is preferably set in the range from 0.8-fold to 3-fold mol with respect to the coloring particles, and more preferably in the range from 1-fold to 2-fold mol with respect to the coloring particles. Although corresponding to a type of the dye used in combination with the metal containing compound, generally if the content of the metal containing compound is set in 0.8-fold mol or more, photoresist performance of the coloring micro particle is greatly increased. Further, dispersion stability of the coloring micro particle is improved by setting the content of the metal containing compound in 3-fold mol or less. This can facilitate the preparation of toner.

(Toner

As the electronic photography toner in an embodiment of the present invention, it is possible to use a known charge control agent and a known offset prevention agent besides the above mentioned thermoplastic resin and the coloring micro particle. Herein, the charge control agent is not specifically limited. For example, a colorless, white or pale colored charge control agent which does not deteriorate a color tone and translucency of the color toner can be used as a negative charge control agent used for color toner. As such a charge control agent, for example, preferably used are a metal complex composed of zinc or chrome with a salicylic acid derivative, a calixarene based compound, an organoboron compound, and a fluorine containing quarternary ammonium salt based compound or the like.

Herein, the following compounds can be used for the above mentioned applications. That is, the salicylic acid metal complexes are described, for example, in JPS53-127726 and JPS62-145255. The calixarene based compounds are described, for example, in JPH02-201378. The organoboron compounds are described, for example, in JPH02-221967. The fluorine containing quarternary ammonium salt based compounds are described, for example, in JFH03-1162.

When those charge control agents are used, it is preferable to use a charge control agent with the content in the range from 0.1 to 10 parts by mass with respect to 100 parts by mass of the thermoplastic resin (or binder resin), and more preferably with the content in the range from 0.5 to 5.0 parts by mass.

Further, the offset prevention agent in an embodiment of the present invention is not specifically limited. For example, the following materials can be used, including a polyethylene wax, an oxidized polyethylene wax, a polypropylene wax, an oxidized polypropylene wax, a carnauba wax, a Southall wax, a rice bran wax, a candelilla wax, a jojoba oil wax, and a beeswax wax or the like. An addition rate of the above mentioned wax is preferably set in the range from 0.5 to 30 parts by mass with respect to 100 parts of mass of the thermoplastic resin (or binder resin), more preferably set in the range from 1 to 20 parts by mass.

Those ranges are set based on the findings that the effect exerted by the addition of the wax becomes insufficient if the addition rate is smaller than 0.5 parts by mass, while translucency and color reproduction of the toner becomes lower if the addition rate is larger than 30 parts by mass.

Further, the compounds described and cited, for example, in JPH08-29934 (pp. 10-13) may be added as an image stabilizer so as to improve storability of the dye. Such a compound is commercially available, for example, including a phenol based compound, an amine based compound, a sulfur based compound and a phosphor based compound or the like. For the same purpose as mentioned above, an ultraviolet absorber may be also added, including an organic ultraviolet absorber and an inorganic ultraviolet absorber.

Such an organic ultraviolet absorber includes a benzotriazole based compound such as 2-(2′-hydroxy-5′-t-butylphenyl)benzotriazole and 2-(2′-hydroxy-3′,5′-di-t-butylphenyl)benzotriazole; a benzophenone based compound such as 2-hydroxy-4-methoxybenzophenone and 2-hydroxy-4-n-octyloxybenzophenone; a hydroxybenzoate based compound such as phenyl salicylate, 4-t-butylphenyl salicylate, 2,5-di-t-butyl-4-hydroxybenzoic acid n-hexadecyl ester and 2,4-di-t-butylphenyl 3′,5′-di-t-butyl-4′-hydroxybenzoate or the like. Further, such an inorganic ultraviolet absorber includes titanium oxide, zinc oxide, cerium oxide, iron oxide, and barium sulfate or the like.

However, the organic ultraviolet absorber is more preferable than the inorganic ultraviolet absorber. Such an ultraviolet absorber preferably has the wavelength in the range from 350 to 420 nm at 50% penetration rate, and more preferably in the range from 360 to 400 nm at 50% penetration rate. If the wavelength is lower than 350 nm, the UV-cut ability becomes weaker. In contrast, if the wavelength is higher than 420 nm, the color of toner becomes deeper, resulting in the undesirable appearance. The addition content of the ultraviolet absorber is not specifically limited. However, the addition content of the ultraviolet absorber is preferably set in the range from 10 to 200 mass % with respect to the dye, and more preferably set the range from 50 to 150 mass % with respect to the dye.

(Binder Resin)

A binder resin contained in the electric photography toner in an embodiment of the present invention is preferably a thermoplastic resin which increases the adhesive property with a coloring micro particle or a micro particle of the copper complex compound, and more preferably a solvent-soluble resin. Further, if a precursor of the resin polymer is solvent-soluble, it is possible to use a thermosetting resin which forms a 3-dimensional structure. Generally a resin used for a binder resin of toner can be used as the thermoplastic resin without specifically limited.

For example, the following resins are preferably used as such a binder resin, including a styrene based resin; an acrylic resin such as an alkyl acrylate and an alkyl methacrylate; styrene acrylic copolymer resin; a polyester based resin; a silicone resin; an olefin resin; an amide resin; and an epoxy resin. Among the above mentioned resins, demanded is a resin having high transparency profile and such melting profile as low viscosity and highly sharp melting. Herein, such a binder resin having the above mentioned profile preferably includes a styrene based resin, an acrylic resin and a polyester based resin.

Further, it is desirable to use the following binder resin. Preferably such a binder resin has a number average molecular weight (Mn) in the range from 3000 to 6000, and more preferably from 3500 to 5500. Moreover, the binder resin preferably has an Mw/Mn rate in the range from 2 to 6, and more preferably from 2.5 to 5.5, the Mw/Mn rate being a rate of a weight average molecular weight (Mw) to a number average molecular weight (Mn). Furthermore, the glass transition temperature of the binder resin is in the range from 50 to 70° C., preferably from 55 to 10° C. The softening temperature thereof is in the range from 90 to 110° C., and preferably from 90 to 105° C.

If the number average molecular weight of the binder resin is smaller than 3000, an image part is detached from an image support when a solid image material with full colors is bended, thereby to cause an image loss (that is, deterioration in the fixing profile when a solid image material is bended). In contrast, if the number average molecular weight of the binder resin is larger than 6000, thermal melting profile thereof at the fixing time is deteriorated, resulting in the decrease in the fixing property. Further, if the rate of Mw/Mn is smaller than 2, the high temperature offset tends to easily occur. However, if the rate of Mw/Mn is larger than 6, the sharp melting profile at the fixing time is deteriorated, resulting in the deterioration in the translucency of the toner and the miscibility of colors when a full color image is formed.

Further, if the glass transition temperature of the binder resin is less than 50° C., heat resistance of the toner becomes insufficient, tending to cause agglomeration of the toner at the storage time. In contrast, if the glass transition temperature of the binder resin is higher than 70° C., the binder resin becomes difficult to melt. This results in the decrease in not only the fixing property but also the miscibility of colors when a full color image is formed. Moreover, if the softening temperature of the binder resin is less than 90° C., high temperature offset is easy to occur. In contrast, if the softening temperature of the binder resin is more than 110° C., the fixing performance, translucency, color miscibility and lustrous property of a full color image are decreased.

The electronic photography toner in an embodiment of the present invention can be produced by using the above mentioned thermoplastic resin (or binder resin), the coloring micro particles and other desired additives (wherein several types of or one type of the micro particles may be mixed). The method for producing the toner includes other known methods such as a kneading method, a grinding method, a suspension polymerization method, an emulsion polymerization method, an emulsifying dispersion granulation method and a capsulation method or the like. Among those production methods, if downsizing of the toner particle size associated with the more improvement in the visualized image is taken in consideration, preferable an emulsion polymerization method may be used from the viewpoint of cost and stability in the production.

Herein, the emulsion polymerization method comprises the steps of mixing thermoplastic resin emulsion produced via the emulsion polymerization with other dispersing liquid of the toner particle component such as the coloring micro particles; slowly agglomerating the produced particles via pH control of the mixed suspension by balancing the repulsion force and the agglomeration force generated by the addition of an electrolyte, on the particle surface; gathering the particles with controlling the particle size and the particle distribution and simultaneously heating and stirring the resultant liquid mixture, thereby to control the fusion between the micro particles and the shaping. As a result, the toner particles are produced by the above mentioned method.

Further, it is preferable to control the volume average particle size of the electronic photography toner particles in an embodiment of the present invention, in the range from 4 to 10 μm, and more preferably from 6 to 9 μm from the viewpoint of highly precise reproduction of the image.

Moreover, it is possible to use the electronic photography toner in an embodiment of the present invention by adding and mixing a post-treatment agent from the viewpoint of adding the flowability for the toner and improving the cleaning performance. Such a post-treatment agent is not specifically limited.

The post-treatment agent includes, for example, inorganic oxide micro particles such as silica micro particles, alumina micro particles and titania micro particles; inorganic stearic acid compound micro particles such as aluminum stearate micro particles and zinc stearate micro particles; inorganic stearic acid compound micro particles such as aluminum stearate micro particles and zinc stearate micro particles; titanic acid compound micro particles such as strontium titanate and zinc titanate or the like. The post-treatment agent can be used alone or in combination with a different kind of additive. Herein, it is preferable to use the above mentioned micro particles after undergoing the surface treatment with a silane coupling agent, a titan coupling agent, a high fatty acid and a silicone oil or the like from the viewpoint of the environment resistant stability and the heat resistant storage stability.

The addition content of the post-treatment agent is used preferably in the range from 0.05 to 5 parts by mass, more preferably from 0.1 to 3 parts by mass with respect to the toner in 100 parts by mass.

Further, it is possible to use the electronic photography toner in an embodiment of the present invention as 2 components developing toner used by mixing a carrier, or 1 component developing toner without using any carrier.

Here, conventionally a known carrier for 2-component development can be used as a carrier used in combination with the electronic photography toner in an embodiment of the present invention. For example, it is possible to use a carrier made of magnetic particles such as iron particles and ferrite particles; a resin coating carrier which is formed by coating those magnetic particles with a resin, or a binder typed carrier which is formed by dispersing those magnetic particles in the binder resin.

Among the above mentioned carriers, a resin of the resin coating carrier is not specifically limited. For example, the following resins are preferably used for a resin coating carrier from the viewpoint of suppression of tonerspent, including an olefin based resin, a styrene based resin, a styrene acrylic copolymer resin, a silicone resin, a copolymer resin made of an organopolysiloxane monomer and a vinyl monomer (or graft resin); and a fluorine resin or a polyester resin. Particularly, the carrier which is coated with the resin produced by the reaction of the copolymer resin made of organopolysiloxane and a vinyl based monomer, with isocyanate is preferable from the viewpoint of the environment resistant stability and tonerspent resistant profile.

Herein, it is needed to use a monomer with a substituent such as a hydroxyl group having reactivity with isocyanate for the vinyl based monomer. Further, the resin composing the resin dispersion typed carrier is not specifically limited and a known resin can be used for such a resin. For example, it is possible to use a styrene acrylic copolymer resin, a polyester resin, a fluorine resin, and a phenol based resin or the like. Further, it is preferable to use a resin having a Volume average particle size in the range from 20 to 100 μm, more preferable from 20 to 60 μm from the viewpoint of securing the high image quality and avoiding the carrier covering. The volume average particle size of the carrier can be measured by a laser diffraction particle size distribution analyzer equipped with a wet type disperser (“HELOS”; SYMPATEC GmbH).

(Method for Forming Image)

Next, a method for forming an image by using the electronic photography toner in an embodiment of the present invention will be explained in detail.

In the embodiment of the present invention, a method for forming an image is not specifically limited. For example, one method comprises the steps of forming a plurality of images on a photosensitive material; and transferring the image onto an image support in a lump. Another method comprises the steps of forming an image on a photosensitive material; and transferring the image one after another onto a transferring belt. As mentioned above, the method is not specifically limited, while the preferable method comprises the steps of forming a plurality of images on a photosensitive material; and transferring the image onto an image support in a lump.

More specifically, that method is conducted comprising the steps of uniformly charging the photosensitive material; exposing the photosensitive material corresponding to a resultant first image; and then firstly developing the material so as to form a first toner image on the photosensitive material. Next, the method is further conducted comprising the steps of uniformly charging the photosensitive material on which the first image has been formed; exposing the photosensitive material corresponding to a resultant second image; and then secondly developing the material so as to form a second toner image on the photosensitive material.

Then, the method is moreover conducted comprising the steps of uniformly charging the photosensitive material on which the first and second images have been formed; exposing the photosensitive material corresponding to a resultant third image; and then thirdly developing the material so as to form a third toner image on the photosensitive material. Finally, the method is furthermore conducted comprising the steps of uniformly charging the photosensitive material on which the first, second and third images have been formed; exposing the photosensitive material corresponding to a resultant fourth image; and then fourthly developing the material so as to form a fourth toner image on the photosensitive material.

In other words, the method is conducted comprising the steps of firstly developing the material by using a yellow toner; secondly developing the resultant material by using a magenta toner; thirdly developing the resultant material by using a cyan toner; and fourthly developing the resultant material by using a black toner.

As a result, a full color toner image is formed on the photosensitive material. After the procedure, the resultant image formed on the photosensitive material is transferred on an image support such as paper in a lump, and the resultant image is fixed on the image support, thereby to form a final image.

In the above mentioned method, the image formed on the photosensitive material is transferred onto paper or the like, thereby to form a final image. Therefore, different from a so-called intermediate transferring method, it is possible to complete a transferring treatment which is a factor of disordering the formed image in only a single process, allowing the image quality to be more increased.

Further, in the above mentioned method, multiple times of development treatments are needed in the method for developing the photosensitive material. Therefore, a non-contact development method is preferable. Alternatively, preferable is also a method for applying an alternating electric field to the material at the development.

Further, as mentioned hereinbefore, a non-contact development method is preferable when the development method comprises the steps of forming a superimposed color image on an image forming material; and transferring the resultant image in a lump.

Further, in order to speed up the procedure, a method is used comprising the steps of arranging a plurality of photosensitive materials corresponding to the respective colors and a development apparatus; successively transferring an image corresponding to the respective colors formed on the plurality of photosensitive materials onto an intermediate transferring material in a superimposed manner: transferring the resultant superimposed image onto an image support such as paper in a lump, thereby to obtain a full color image.

In the above mentioned method, a contact development method can be applied as a development procedure. Herein, it is possible to use both a one-component developer and a two-component developer as a developer. This kind of the method is called a tandem method, and used for a high speed machine since both a monochrome image and a full color image can be formed at the same speed by one-time exposure.

A preferable fixing method used in an embodiment of the present invention includes a so-called contact heating method. In particular, a representative contact heating method includes a heat roll fixing method and a heat-pressure fixing method for fixing an image via using a rolling pressure member which has a heating unit arranged in a fixed positioning manner.

(Image)

In an image forming process which is comprised of developing, transferring and fixing steps by using the electronic photography toner in an embodiment of the present invention, the following state is shown from the transferring step to the fixing step.

That is, as to the electronic photography toner in the embodiment of the present invention, the coloring micro particle in the toner transferred onto a transferring material does not collapse after the fixing step and adheres to paper at a dispersed state in a toner particle.

In an embodiment of the present invention, the dye is not separated (or does not migrate) from the inside of a toner particle into a surface thereon due to the dispersion condition of the coloring micro particles inside the toner particle as described hereinbefore, although the toner particle includes the dye with high density.

Therefore, the following drawbacks in the prior art can be avoided. That is, are shown the drawbacks of the toner on which surface a dye is exposed, the dye being obtained by dispersing or dissolving the dye into a thermoplastic resin (or toner binder resin) as it is. Such drawbacks include: 1) a low charge amount; 2) a large difference of the charge amounts under the conditions between a high temperature and high humidity status and a low temperature and low humidity status (or environmental dependence); and 3) a large deviation of the charge amount for the respective color toners when such a pigment as cyan, magenta, yellow or black pigment is used as in the case of full color image record, in other words, a large deviation of the charge amount for the toners corresponding to the kinds of the coloring agents therein.

Further, when the toners are heat fixed on a transferring material, the dye which is a coloring agent does not migrate into the outside of the coloring micro particle (or exposure on a surface of the coloring micro particle). Therefore, this can prevent the dye from subliming or the oil contamination at the time in the heat fixing step. Such drawbacks are issues on the tones using a generally used dye therein.

As described hereinbefore, conventional drawbacks have been solved by the method for producing cyanoacetic acid in an embodiment of the present invention, the method comprising the step of hydrolyzing a cyanoacetate with a predetermined chemical formula in the presence of an acid catalyst.

In other words, an amount of malonic acid produced as a by-product can be greatly decreased by controlling the balance between the residual amount of the cyanoacetate at the end point of the hydrolysis reaction and the residual content of the alcohol produced during the hydrolysis reaction. Further, the cyanoacetic derivative produced by cyanoacetic acid as a starting material which has been prepared via the above described method together with the metal containing compound prepared by using the cyanoacetic acid derivative have greatly improved purity as well as yields. Eventually, a production method excellent in the producibility and cost can be found out and realized.

### EXAMPLES

Hereinafter, Examples in the embodiments of the present invention will be explained in detail. However, the present invention is not specifically limited to such Examples.

### Example 1

### Preparation of Cyanoacetic Acid

To a three-necked flask, were added ethyl cyanoacetate (20 g, 0.1768 mol) and pure water (100 ml) following p-toluenesulfonic acid monohydrate (1.7 g, 5 mol %), and the resultant reaction mixture was refluxed for 4 hr. Then, ethanol and water in the reaction mixture were removed by distillation using a Dean-Stark apparatus, while the reaction mixture was simultaneously heated for 6 hr. After completion of the reaction, the resultant aqueous reaction mixture was condensed under a reduced pressure at 60 to 70° C., thereby to yield a 6.0% cyanoacetic acid aqueous solution.

(Analysis and Evaluation)

The reaction was traced by gas chromatography (GL Sciences Inc.: GC390). In the analysis, the calibration curve data on ethyl cyanoacetate and cyanoacetic acid were checked, and the end point of the hydrolysis reaction was set at the time when the content of ethyl cyanoacetate substantially reached 2 mol %. Similarly, the contained ethanol amount at the end point was also quantitatively measured by gas chromatography. The content of malonic acid in a test sample was analyzed by ion chromatography (Japan Dionex Inc.; Ion Chromatograph DX-500) based on the calibration curve generated by the commercially available reference standard.

### Examples 2-13

Following the same procedure as in Example 1, in Examples 2-13, an acid catalyst and an amount thereof as well as an ethanol amount at the end point of the hydrolysis reaction were variously modified.

### Comparative Examples 1-5

Comparative Examples 1-5 were conducted following the same procedure as in Example 1, except that the hydrolysis reaction was continued until cyanoacetate was substantially incapable of being detected, as extending the end point of the reaction.

Table 1 shows the above mentioned results.

TABLE 1

| | | | Acid Catalyst | | | | | |
|---|---|---|---|---|---|---|---|---|
| | | | Content | | | Residual | Containing | |
| | | | (mol % per | Reaction | Residual Ethanol | Cyanoacetate | Malonic Acid | |
| | Acid Catalyst | Cyanoacetate | cyanoacetate) | Time (h) | Content (mol %) | Content (mol) | Content (mol %) | Note |
| Example 1 | a) | Ethyl Cyanoacetate | 5.0 | 6 | 5.7 | 2.1 | 0.89 | b) |
| Example 2 | a) | Ethyl Cyanoacetate | 10.0 | 16 | 51.6 | 2 | 0.97 | b) |
| Example 3 | a) | Methyl Cyanoacetate | 2.0 | 19 | 26.4 | 1.8 | 0.83 | b) |
| Example 4 | a) | Ethyl Cyanoacetate | 0.5 | 20 | 41.2 | 2.2 | 0.86 | b) |
| Example 5 | a) | Ethyl Cyanoacetate | 0.5 | 12 | 9.5 | 1.2 | 0.77 | b) |
| Example 6 | a) | Ethyl Cyanoacetate | 0.5 | 16 | 18.5 | 1.9 | 0.81 | b) |
| Example 7 | a) | Ethyl Cyanoacetate | 0.5 | 10 | 12.3 | 1.4 | 0.64 | b) |
| Example 8 | a) | Ethyl Cyanoacetate | 0.5 | 7 | 14.8 | 1.7 | 0.55 | b) |
| Example 9 | Hydrochloric Acid | Ethyl Cyanoacetate | 0.5 | 11 | 11.6 | 1.8 | 0.86 | b) |
| Example 10 | Sulfuric Acid | Methyl Cyanoacetate | 0.5 | 9 | 15.8 | 1.6 | 0.89 | b) |
| Example 11 | Phosphoric Acid | Ethyl Cyanoacetate | 0.5 | 13 | 21.2 | 1.5 | 0.79 | b) |
| Example 12 | Acetic Acid | Ethyl Cyanoacetate | 0.5 | 14 | 13.1 | 1.7 | 0.81 | b) |
| Example 13 | Cyanoacetic Acid | Ethyl Cyanoacetate | 0.5 | 15 | 10.6 | 1.9 | 0.76 | b) |
| Comparative | a) | Ethyl Cyanoacetate | 5.0 | 8 | 0.3 | 0 | 1.83 | c) |
| Example 1 | | | | | | | | |
| Comparative | a) | Ethyl Cyanoacetate | 0.5 | 12 | 0.2 | 0.1 | 1.24 | c) |
| Example 2 | | | | | | | | |
| Comparative | a) | Methyl Cyanoacetate | 0.5 | 15 | 0.15 | 0 | 1.48 | c) |
| Example 3 | | | | | | | | |
| Comparative | Hydrochloric Acid | Ethyl Cyanoacetate | 5.0 | 10 | 0.25 | 0.1 | 2.56 | c) |
| Example 4 | | | | | | | | |
| Comparative | nitric acid | Ethyl Cyanoacetate | 0.5 | 14 | 0.18 | 0 | 2.38 | c) |
| Example 5 | | | | | | | | |

a) p-toluenesulfonic acid monohydrate;
b) Present Invention;
c) Comparative Invention

The results in Table 1 demonstrate that cyanoacetic acid prepared by the method in an embodiment of the present invention includes the greatly decreased content of malonic acid produced as a byproduct. Herein, at the end point of the hydrolysis reaction the same as in Example 8, when the residual content of the cyanoacetate was at a level of 4.8 mol % (where the residual ethanol content=12.8%; reaction time=5.5 h) or 0.7 mol % (where the residual ethanol contents 0.2%; reaction time=8 h), the malonic acid content included in the corresponding reaction mixture was 0.49 mass % or 0.65 mass %.

It should be noted that when the residual ethanol content was 60.8 mol %, the residual content of ethyl cyanoacetate was 6.7 mol %. If the residual content of ethyl cyanoacetate is increased, this may result in the increase in the impurity content in the later step of preparing a cyanoacetic acid derivative. This drawback is not preferable for the actual manufacturing.

### Comparative Example 6

### Method Described in WO2011/010509A1

<Preparation of Cyanoacetic Acid Derivative and Metal containing Compound>

Here, compounds B and C which were cyanoacetic acid derivatives, and an exemplary compound 5 which was a metal containing compound were prepared by the following method.

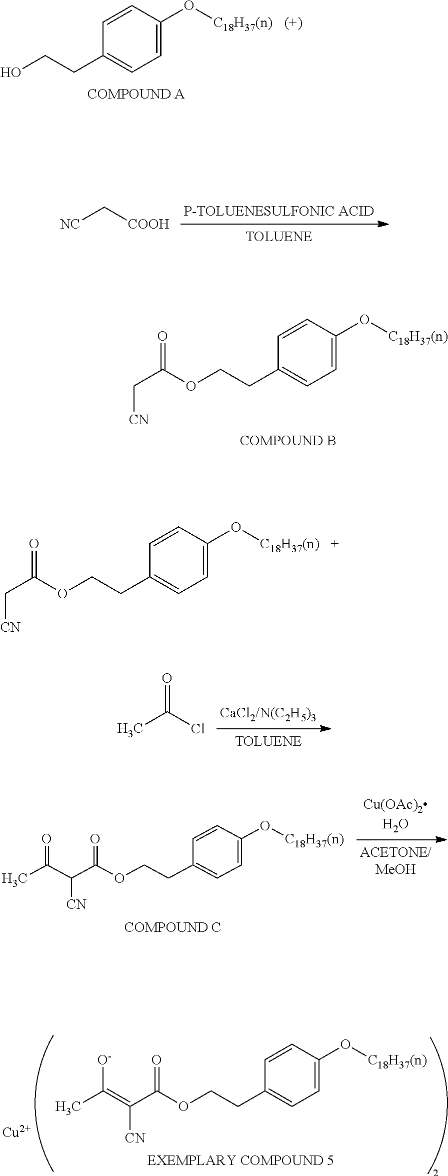

Preparation of Compound B (Second Step)

To a three-necked flask (500 ml), were added a compound A (90 g), cyanoacetic acid (21.5 g), p-toluenesulfonic acid monohydrate (1.31 g), and toluene (300 ml). By using an esterification tube (or Dean-Stark apparatus) to remove water, the reaction mixture was heated to be refluxed for 2 h. After the solvent was removed by distillation under a reduced pressure, acetone (500 ml) was added for recrystallization, thereby to yield a compound B in 89.5% (94.4 g). Note a by-product compound (referred to as DD4M) of the formula (3′) was observed in 0.65% as a simple area ratio, when the reaction was traced by HPLC analysis.

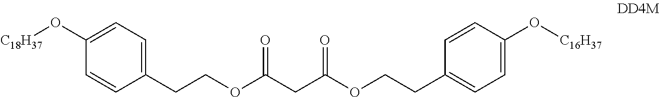

Preparation of Compound C (Third Step)

To a three-necked flask (100 ml), were added the compound B (5 g), toluene (25 ml), triethylamine (3.3 g), and calcium chloride (2.42 g), and the reaction mixture was heated to 80° C. with stirring. After the inside temperature reached 80° C., acetyl chloride (2.1 g) was dropwisely added over 1 h. Then, after the completion of the dropping, the reaction mixture was cooled, washed with a diluted hydrochloric acid aqueous solution and neutralized the pH value by pure water, and the solvent was removed by evaporation. To the resulting residue, were added toluene (50 ml) and ethyl acetate (50 ml) for recrystallization, to yield a compound C in 78.8% (4.3 g).

¹H-NMR (CDCl₃): δ=0.88 (t, 3H), 1.20-1.28 (m, 28H), 1.42 (m, 2H), 1.76 (m, 2H), 2.13 (s, 3H), 3.01 (t, 2H), 3.93 (t, 2H), 4.48 (t, 2H), 6.87 (d, 2H), 7.19 (d, 2H), 14.17 (s, 1H).

Preparation of Exemplary Compound 5 (Fourth Step)

To a three-necked flask (200 ml), were added the compound C (2 g) and acetone (80 ml), and the reaction mixture was heated until the inside temperature reached 55° C. with stirring. Then, copper acetate monohydrate (0.55 g) was dissolved in a mixed solvent (5 ml; MeOH/water=5/1) and the resulting solution was dropwisely added to the reaction mixture over 30 min. After the completion of the dropping, the precipitated solid was filtered, to yield an exemplary compound 5 in 65.9% (1.4 g). MP: 146-147° C. Table 2 shows the results. In Comparative Example 6, a total yield from the second step to the fourth step was 46.5%.

### Comparative Example 7

The exemplary compound 5 was prepared in the same manner as in Comparative Example 6 by using cyanoacetic acid produced in Comparative Example 1. Note all the preparation steps were conducted through a running manner without conducting the recrystallization as in Comparative Example 6. Table 2 shows the results. Here, the number of each step in Table 2 shows a reaction rate calculated based on the calibration curve in the HPLC analysis. In the second step, the byproduct (DD4M) was observed in 4.4% and the total yield from the second step to the fourth step was 78.8%.

### Example 14

The exemplary compound 5 was prepared in the same manner as in Comparative Example 6 by using cyanoacetic acid produced in Example 8. Note, all the preparation steps were conducted through a running manner without conducting the recrystallization the same as in Comparative Example 7. Here, the number of each step in Table 2 shows a reaction rate calculated based on the calibration curve in the HPLG analysis. In the second step, the byproduct (DD4M) was observed in 1.6% and the total yield from the second step to the fourth step was 82.9%.

### Example 15

The exemplary compound 5 was prepared in the same manner as in Comparative Example 6 by using cyanoacetic acid produced in Example 8. Note all the preparation steps were conducted through a running manner without conducting the recrystallization the same as in Comparative Example 7 and Example 14. Here, the different features from Example 14 will be described below.

(Third Step)

The reaction was conducted by using 3-fold mol of triethylamine per the compound B and 1.1-fold mol of acetic acid anhydride instead of acetyl chloride per the compound B, without using calcium chloride, at 40-50° C. for 8 h.

(Fourth Step)

The reaction was conducted by using toluene-methanol mixed solvent as a reaction solvent, 0.55-fold mol of copper chloride monohydrate instead of copper acetate per the compound C, and 0.95-fold mol of sodium methoxide per the compound C, Table 2 shows the results. Note the number of each step in Table 2 indicates the reaction rate calculated based on the calibration curve in the HPLC analysis. In the second step, the byproduct (DD4M) was observed in 1.6% and the total yield was 94.0%.

### Comparative Example 8

The reactions undergone from the second step to the fourth step were conducted in the same manner as in Example 15 by using cyanoacetic acid prepared in Comparative Example 5. Table 2 shows the results. Note the number of each step in Table 2 indicates the reaction rate calculated based on the calibration curve in the HPLC analysis. In the second step, the byproduct (DD4M) was observed in 5.8% and the total yield was 86.6%.

TABLE 2

| | | Yields in Each Step | | | | | |
|---|---|---|---|---|---|---|---|
| | Cyanoacetic | (Reaction Rate) (%) | | | | | |
| | Acid | 2nd Step | a) | 3rd Step | 4th Step | b) | Note |
| Comparative | Agent | 89.5 | 0.65 | 78.8 | 65.9 | 46.5 | Comparative |
| Example 6 | | | | | | | Example |
| Comparative | Comparative | 94.3 | 4.4 | 95.2 | 87.8 | 78.8 | Comparative |
| Example 7 | Example 1 | | | | | | Example |
| Example 14 | Example 8 | 97.2 | 1.6 | 96.3 | 88.6 | 82.9 | Present |
| | | | | | | | Invention |
| Example 15 | Example 8 | 97.2 | 1.6 | 98.4 | 98.3 | 94.0 | Present |
| | | | | | | | Invention |
| Comparative | Comparative | 90.3 | 5.8 | 97.8 | 98.1 | 86.6 | Comparative |
| Example 8 | Example 5 | | | | | | Example |

a) DD4M Content (HPLC simple area rate;
b) Total Yield (2nd step-4th step) (%)

The results in Examples 14 and 15, and Comparative Examples 6-8 demonstrated that when cyanoacetic acid produced by the method of the present invention, the content of the byproduct generated in the method for producing the cyanoacetic acid derivative and the metal containing compound in the later step was greatly reduced. Further, it was demonstrated that the running yield over all the steps was highly improved.

### Examples 16-22

Exemplary compounds 8, 11, 15, 17, 22, 24 and 27 were prepared by the same method as in Example 15.

Table 3 shows the results. Note the purity listed in Table 3 represents an average value calculated through the resulting data obtained by ICP of quantitatively analyzing copper content in the sample after the preparation and drying of the exemplary compound, and the theoretical value (N−5), as shown in the following formula.

Purity (%)=(Quantitative Analytical Result of Copper by ICP)/(Theoretical Value of Copper Content of Compound)×100

(Average Value Calculated via Five Times Measurements)

Similarly, Table 3 shows the results in Comparative Examples 6-8.

TABLE 3

| | Compound No. | a) | Purity (%) | Note |
|---|---|---|---|---|
| Example 15 | 5 | 94.0 | 99.8 | Present Invention |
| Example 16 | 8 | 91.5 | 99.6 | Present Invention |
| Example 17 | 11 | 92.7 | 99.9 | Present Invention |
| Example 18 | 15 | 94.8 | 99.7 | Present Invention |
| Example 19 | 17 | 90.1 | 99.8 | Present Invention |
| Example 20 | 22 | 92.2 | 99.6 | Present Invention |
| Example 21 | 24 | 93.5 | 99.9 | Present Invention |
| Example 22 | 27 | 91.9 | 99.7 | Present Invention |
| Comparative | 5 | 46.5 | 99.7 | Comparative |
| Example 6 | | | | Example |
| Comparative | 5 | 78.8 | 98.2 | Comparative |
| Example 7 | | | | Example |
| Comparative | 5 | 86.6 | 98.5 | Comparative |
| Example 8 | | | | Example |

a) Running (or Total) Yields from 2nd Step to 4th Step (%)

Further, in the method of Example 15, when treatments of the re-heating, water washing, condensing and recrystallization in a toluene-methanol mixed solvent were conducted as a post-treatment in the fourth step, the total yield was slightly lowered as in 92.7%, while the purity was improved up to 99.95%.

The above mentioned results demonstrated that the method for producing the cyanoacetic acid derivative and the metal containing compound, via using cyanoacetic acid prepared in the method of the present invention enabled the side-reaction to be sufficiently suppressed and the final product to be obtained without requiring the purification process in the representative steps.

Accordingly, the method of the present invention allows the total yields of cyanoacetic acid, the cyanoacetic acid derivatives and the metal containing compounds to be greatly improved, leading to finding of the preparation method excellent in sufficiently economical performance.

As mentioned hereinbefore, the method for producing cyanoacetic acid of the present invention allows the content of malonic acid generated as a byproduct to be largely reduced, resulting in the improvement in the quality of the produced cyanoacetic acid. Thus, this outstanding effect results in the improvement in the quality of the cyanoacetic acid derivative and the metal containing compound, which are prepared from the cyanoacetic acid thus produced having the high quality as the starting material.

Further, the method for producing cyanoacetic acid of the present invention enables the purity and the yields of both cyanoacetic acid derivative and metal containing derivative to be greatly improved.

Therefore, those advantageous effects exerted by the method of the present invention significantly enhance the productivity and the economical efficiency in the production of the above target compounds.

What is claimed is:

1. A method for producing cyanoacetic acid, comprising the step of hydrolyzing a cyanoacetate of a formula (1) in the presence of an acid catalyst in a reaction mixture thereby to obtain cyanoacetic acid as well as an alcohol and a malonic acid generated as a byproduct,
wherein when the hydrolysis reaction is completed,
the cyanoacetate of the formula (1) is included in 0.5 to 5 mol % with respect to the produced cyanoacetic acid;
the alcohol is included in 0.5 to 60 mol % with respect to the produced cyanoacetic acid; and
the malonic acid byproduct is included in 1.0 mol % or less with respect to the produced cyanoacetic acid, in the reaction mixture, 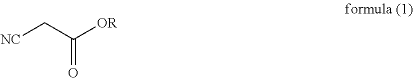
[where R represents an ethyl group or a methyl group].

2. The method for producing cyanoacetic acid according to claim 1, the alcohol produced in the hydrolysis reaction is included in 0.5 to 20 mol % with respect to the produced cyanoacetic acid, when hydrolysis reaction is completed.

3. The method for producing cyanoacetic acid according to claim 1, the cyanoacetate of the formula (1) is included in 0.5 to 2.0 mol % with respect to the produced cyanoacetic acid, when the hydrolysis reaction is completed.

4. The method for producing cyanoacetic acid according to claim 1, wherein the acid catalyst is selected from a group of sulfuric acid, hydrochloric acid, acetic acid, cyanoacetic acid, phosphoric acid, and p-toluenesulfonic acid.

5. The method for producing cyanoacetic acid according to claim 1, wherein the acid catalyst is used in 0.2 to 10 mol % with respect to the cyanoacetate of the formula (1) in the hydrolysis reaction.

6. The method for producing cyanoacetic acid according to claim 5, wherein the acid catalyst is used in 0.2 to 2.0 mol % with respect to the cyanoacetate of the formula (1) in the hydrolysis reaction.

7. The method for producing cyanoacetic acid according to claim 1, further including the step of removing the alcohol produced in the hydrolysis reaction via distillation.

8. The method for producing cyanoacetic acid according to claim 7, further including the step of reflux prior to removing the alcohol produced in the hydrolysis reaction via distillation.

9. A method for producing a cyanoacetic acid derivative of a formula (3), comprising the step of:
undergoing a reaction of a compound of a formula (2) with cyanoacetic acid produced in the method as described in claim 1 thereby to produce a cyanoacetic acid derivative of a formula (3),
HO—R₁ formula (2)
[where R₁ represents a group having an aromatic hydrocarbon structure containing nine or more carbon atoms], and 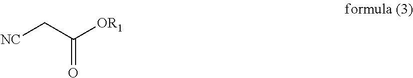
[wherein R₁ represents the group having the aromatic hydrocarbon structure containing nine or more carbon atoms].

10. A method for producing a metal containing compound of formula (5); comprising the step of:
undergoing a reaction of the compound of the formula (3) produced in the method as described in claim 9 with an acid chloride or an acid anhydride thereby to produce a compound of a formula (4); and
undergoing subsequently a reaction of the compound of the formula (4) with copper chloride or copper acetate, thereby to produce the metal containing compound of a formula (5), 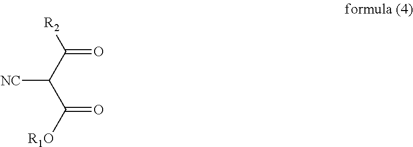
[where R₁ represents the group having the aromatic hydrocarbon structure containing nine or more carbon atoms, and R₂ represents an alkyl group], and 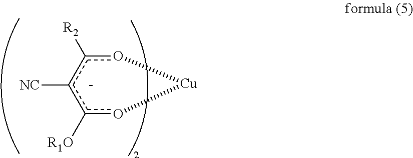
[where R₁ represents the group having the aromatic hydrocarbon structure containing nine or more carbon atoms, and R₂ represents the alkyl group].

11. The method for producing the metal containing compound of the formula (5) as described in claim 10, wherein
the metal containing compound of the formula (5) is produced without purifying the compound of the formula (3) and the compound of the formula (4).
